(19) United States
(12) Patent Application Publication
(10) Pub. No.: US 2024/0140935 A1
(43) Pub. Date: May 2, 2024

(54) NON-COVALENT DIMER CATION, SALT THEREOF, PREPARATION METHOD THEREOF, AND USE THEREOF AS ANTIOXIDANT

(71) Applicant: SUZHOU PROTONATION CHAIN BIOTECHNOLOGY CO., LTD., Suzhou (CN)

(72) Inventors: Zizhen ZHAO, Xi’an (CN); Zhigang WANG, Xi’an (CN)

(21) Appl. No.: 18/475,538

(22) Filed: Sep. 27, 2023

(30) Foreign Application Priority Data
Sep. 27, 2022 (CN) 2022111849609

(51) Int. Cl. C07D 403/12; A61P 39/06; C07D 403/14

(52) U.S. Cl. CPC C07D 403/12; A61P 39/06; C07D 403/14; C07B 2200/07

(57) ABSTRACT

Herein provides a non-covalent dimer cation of formula (A), a tautomer thereof, or a stereoisomer thereof. Herein provides a salt, which includes the cation of the present application and a first anion. Herein provides use of the cation of the present application or the salt of the present application in antioxidation, preparation of an antioxidant, preparation of a drug for inhibiting, reducing or reversing oxidative stress in human or animal cells, or preparation of a drug for treating an oxidative stress-associated disease or symptom, such as, use in preparation of a drug for treating non-alcoholic fatty liver disease.

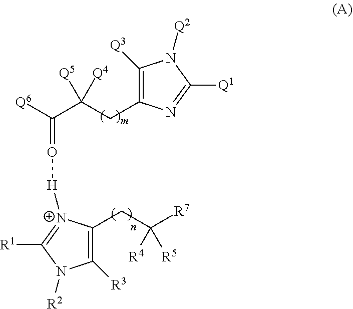

### CROSS-REFERENCE TO RELATED APPLICATIONS

This application claims the priority to Chinese Patent Application No. 202211184960.9, filed to China National Intellectual Property Administration on Sep. 27, 2022, which is incorporated by reference herein in its entirety.

### FIELD

The present application relates to the field of biological medicines, and more specifically, to a non-covalent dimer cation, a salt thereof, a preparation method thereof, and use thereof as an antioxidant, such as, in treatment of non-alcoholic fatty liver disease.

### BACKGROUND

Energy metabolism in an organism takes oxygen as an electron acceptor in the aerobic metabolism process, which inevitably produces reactive oxygen species. Reactive oxygen species have double effects, which are closely associated with the regulation of certain physiologically active substances and the inflammatory immune process. However, excess reactive oxygen species tend to lead to oxidative stress. Mitochondrial respiratory chain complexes use electrons to transfer produced adenosine triphosphate, which are a main source of reactive oxygen species. Liver contains abundant mitochondria, and thus is the main organ that will be attacked by reactive oxygen species. Therefore, oxidative stress may be the common pathogenesis of hepatopathy.

Fatty liver disease refers to abnormal deposit of fat in liver cells, and can be divided into non-alcoholic fatty liver disease and alcoholic fatty liver disease. In the complex pathogenesis of fatty liver, oxidative stress plays a key role. Currently, the widely accepted theory for the pathogenesis of fatty liver is deposit of fat in liver cells due to insulin resistance and imbalance of fat metabolism, in particular fatty acids and triglycerides, which will eventually cause simple hepatic steatosis. The second one is that environmental stressors (dietary ingredients) and metabolic stressors (such as high blood glucose) produce oxidative stress through damage to liver cell mitochondria, which will cause steatohepatitis under the coaction of cytokines, further form fatty liver fibrosis and fatty liver cirrhosis, and may further develop into liver cancer.

At present, treatment of this disease is mainly based on adjusting patient's lifestyle, and drugs are used as an assistant treatment means. For example, vitamin E can improve the total antioxidant capacity of a mouse liver tissue and the glutathione peroxidase activity, and reduce hepatocellular injury. However, the effect of vitamin E is limited, and safety of long-term administration cannot be ensured. Lipid-regulating drugs block the digestion and absorption of fats in the gastrointestinal tract to relieve fatty liver disease. However, fatty substances are one of the necessary substances for human life activities. Therefore, this type of drug usually brings other health risks. The foregoing two types of drugs can only delay the progress of fatty liver disease, and cannot repair fatty liver damaged cells and repair fatty liver damage.

### SUMMARY

The present application provides a non-covalent dimer cation, which is a cation of formula (A), or a tautomer thereof, or a stereoisomer thereof,

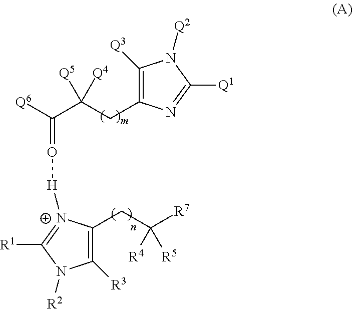

wherein the bond “-------” is a non-covalent bond; R⁷ is selected from a group consisting of hydrogen, hydroxymethyl, —C—R⁶, and —C(═O)—R⁶; R¹ and Q¹ are each independently selected from a group consisting of hydrogen, hydroxyl, sulfhydryl, amino, methyl, ethyl, and phenyl; R² and Q² are each independently selected from a group consisting of hydrogen, methyl, ethyl, propyl, phenyl, benzyloxymethyl, and triphenylmethyl; R³ and Q³ are each independently selected from a group consisting of hydrogen, hydroxyl, sulfhydryl, amino, methyl, ethyl, and phenyl; R⁴ and Q⁴ are each independently selected from a group consisting of hydrogen, methyl, ethyl, and propyl; R⁵ and Q⁵ are each independently selected from a group consisting of hydrogen, benzyl, amino, methylamino, hydrazinyl, trimethylammonium, pyrrolyl, and a substituent derived from an amino group further bonded with the C-terminal carbon atom of an amino acid, dipeptide, or tripeptide; R⁶ and Q⁶ are each independently selected from a group consisting of hydroxyl, oxido, methoxy, and a substituent derived from the N-terminal nitrogen atom of an amino acid, dipeptide, or tripeptide; and m and n are each independently selected from a group consisting of 0 and 1.

The present application provides a salt, which comprises the cation of the present application, and a first anion comprising at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion.

The present application provides a preparation method of the cation of the present application or the salt of the present application, comprising: contacting a first reactant with a first acid to obtain a first acid salt of the first reactant; contacting the first acid salt of the first reactant with a first salt comprising a first anion in an acidic environment to obtain a first solution comprising a protonate cation of the first acid salt of the first reactant and the first anion; adding a second reactant to the first solution in a strongly reductive and acidic environment to obtain a second solution; and adding a first base to the second solution; wherein, the first reactant comprises a compound of formula (C), and the second reactant comprises a compound of formula (D),

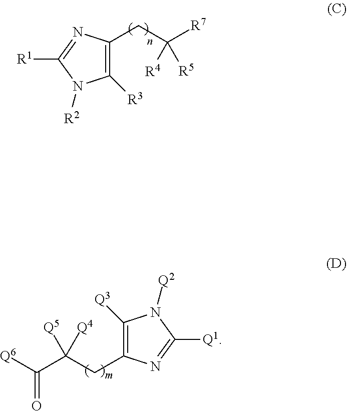

The present application provides use of the cation of the present application or the salt of the present application, in antioxidation, preparation of an antioxidant, preparation of a drug for inhibiting, reducing or reversing oxidative stress in human or animal cells, or preparation of a drug for treating an oxidative stress-associated disease or symptom.

### BRIEF DESCRIPTION OF DRAWINGS

FIG. 1 shows results of oxygen species scavenging experiments of hydrogen peroxide models of some embodiments of the present application.

FIG. 2 shows results of oxygen species scavenging experiments of 2-phenyl-4,4,5,5-tetramethylimidazoline-1-oxyl 3-oxide (PTIO) models of some embodiments of the present application.

FIG. 3 shows cell viability of palmitate non-alcoholic fatty liver cell models of some embodiments of the present application.

FIG. 4 shows levels of reactive oxygen species (ROS), malondialdehyde (MDA), glutathione (GSH), superoxide dismutase (SOD), and adenosine triphosphate (ATP), respectively, in cells of palmitate non-alcoholic fatty liver cell models of some embodiments of the present application. Wherein, part (a) of FIG. 4 shows the level of reactive oxygen species, part (b) of FIG. 4 shows the level of malondialdehyde, part (c) of FIG. 4 shows the level of glutathione, part (d) of FIG. 4 shows the level of superoxide dismutase (SOD), and part (e) of FIG. 4 shows the level of adenosine triphosphate.

FIG. 5 shows cell viability of carbon tetrachloride hepatotoxicity cell model of some embodiments of the present application.

FIG. 6 shows levels of reactive oxygen species, malondialdehyde, glutathione, superoxide dismutase, and adenosine triphosphate, respectively, in cells of carbon tetrachloride hepatotoxicity cell model of some embodiments of the present application. Wherein, part (a) of FIG. 6 shows the level of reactive oxygen species, part (b) of FIG. 6 shows the level of malondialdehyde, part (c) of FIG. 6 shows the level of glutathione, part (d) of FIG. 6 shows the level of superoxide dismutase, and part (e) of FIG. 6 shows the level of adenosine triphosphate.

### DETAILED DESCRIPTION

As used herein, the singular terms refer to singular or plural forms. For example, “an element” or “one element” refer to one or more elements. As used herein, the term “a plurality of” refers to at least two.

As used herein, the term “about” refers to approximately, in the range of about or around. When used in combination with a value range, the term “about” modifies the range by extending the limit above or below the value provided. In general, the term about is used herein to give a value plus or minus 10% from the value provided. In one aspect, the term “about” refers to plus or minus 20% of a value of the number defined by it. For example, “about 50%” refers to a range of 45% to 55%. Herein, a value range defined by endpoints includes all integers and fractions within the range (for example, a range “1 to 5” includes 1, 1.5, 2, 2.75, 3, 3.90, 4, and 5). It should also be understood that all the integers and fractions are defined by the term “about”.

As used herein, the term “include”, “comprise” or “contain”, as a non-exclusive or open-ended term, is intended to refer to a combination (such as a device, a composition or a method) includes listed elements (such as units of the device, components of the composition, or substantive steps of the method), while other elements are not excluded. As used herein, the term “essentially consist of” refers to, when used for defining a composition or a method, exclusion of other elements that have any material effect on the combination of the stated objectives, but does not exclude other elements that do not materially affect the basic and novel features of the present application. As used herein, unless otherwise described, the term “consist of” refers to the exclusion of the combination of other elements (units, components, substantive steps or the like), and is not intended to exclude trace amounts of unavoidable impurities. Embodiments defined by each of these connection terms fall within the scope of the present application. A disclosed technical solution that is defined by the term “include”, “comprise” or “contain” shall be regarded as also disclosing a corresponding technical solution defined by the term “essentially consist of” or “consist of” as a specific embodiment.

As used herein the term “and/or” refers to and covers any or all possible combination of one or more associated listed items. When used in a list of two or more items, the term “and/or” refers to that, any one of the listed items may be included alone, or any combination of two or more of the listed items may be included. For example, if a group, combination, composition or the like is described as comprising (or including) components A, B, C and/or D, the composition may comprise A alone; B alone; C alone; D alone; the combination of A and B; the combination of A and C; the combination of A and D; the combination of B and C; the combination of B and D; the combination of C and D; the combination of A and B and C; the combination of A and B and D; the combination of A and C and D; the combination of B and C and D; or the combination of A and B and C and D.

As used herein, the compound or ion of the present application includes a plurality of variable groups. Those of ordinary skill in the art shall recognize that combinations of the groups that are contemplated by the present application are chemically allowed combinations of compounds or ions.

As used herein, the stereochemistry of a chiral center may be defined according to the conventions of those skilled in the art. That is, the wedged bond “ [symbol] ” is used for indicating a group in front of the plane of the paper (oriented towards the viewer), and the hashed bond “ [symbol] ” is used for indicating a group behind the plane of the paper (oriented away from the viewer). It may be understood that such representations are used for indicating a specific single stereoisomer of groups represented by each chemical structure herein. Any bond that is not specifically represented by the wedged bond or the hashed bond herein shall be regarded as not specifically indicating whether the bond is in front of the plane of the paper, or behind the plane of the paper, or located in the plane of the paper. However, it does not exclude that the bond is in front of or behind the plane of the paper if chemically allowed.

As used herein, the term “isomer” refers to compounds with the same molecular formula and different bonding nature or order of atoms or different arrangements of atoms in space. Wherein, the term “stereoisomer” refers to isomers with different arrangements of atoms in space; the term “enantiomer” refers to stereoisomers with one or more asymmetric centers that are non-superimposable mirror images of each other; the term “diastereomer” refers to stereoisomers that have opposite configurations of one or more asymmetric centers and that do not belong to enantiomers. If a compound has an asymmetric center, for example, if a carbon atom is bonded to four different groups, a pair of enantiomers may exist. A configuration of an enantiomer may be characterized and designated as the R-configuration or the S-configuration by the absolute configuration of one or more asymmetric centers of the enantiomer, or the enantiomer is designated as dextrorotatory or levorotatory based on the manner in which the molecule rotates the plane of polarized light. A chiral compound may exist in the form of an enantiomer alone or a mixture of enantiomers, for example, exist in the form of a racemic mixture. The compound of the present application may have an asymmetric center or chiral center, and exist in the form of different stereoisomers. It should be regarded that all stereoisomers of the compound of the present application include, but are not limited to, a diastereomer, an enantiomer, an atropisomer, and a mixture thereof, such as a racemic mixture, which form part of the present application.

As used herein, the term “dimer” usually refers to a compound formed by two molecules or ions, such as an amino acid, a peptide, or a derivative or protonated ion thereof, through an interaction, in particular a non-covalent interaction. As used herein, the term “covalent bond” refers to any bond that includes or involves electron sharing. Covalent bonds include, but are not limited to, a peptide bond, a glycosidic bond, an ester bond, a phosphodiester bond. As used herein, the term “non-covalent bond” includes any bond or interaction between two or more moieties that do not include or involve electron sharing. Non-covalent bonds or interactions include, but are not limited to, static electricity, π-π effects, the van der Waals force, a hydrogen bond, and the hydrophobic effect, in particular the hydrogen bond.

As used herein, any amino acid with chirality shall be regarded as at least referring to L-amino acid in a case that the chirality is not explicitly described, but it does not explicitly exclude relevant embodiments of D-amino acid.

As used herein, in particular, the term “disclosure or inclusion of” as described below, includes but is not limited to the disclosure of the specification (such as, embodiments), drawings, or claims of the present application, and includes but is not limited to the inclusion in the scope of the claims of the present application, or any one or more embodiments (such as, embodiments defined by the term “in an embodiment” or “in some embodiments” herein).

In order to further describe the technical means adopted by the present application to achieve the intended objective and functions, the following describes specific embodiments, structures, features, and functions of the present application in detail with reference to the accompanying drawings and preferred embodiments.

### Non-Covalent Dimer Cation of the Present Application

In an aspect, the present application provides a non-covalent dimer cation.

The present application provides a non-covalent dimer cation, comprising a moiety of formula (A1) and a moiety of formula (A2), wherein the hydrogen on the imidazole ring in the moiety of formula (A1) (N^{π}-H, i.e. the H marked with an asterisk in formula (A1); or N^{τ}-H under the condition that R² is hydrogen) forms a non-covalent bond with a more electronegative or most electronegative atom in the moiety of formula (A2), such as oxygen (i.e. O marked with an asterisk in formula (A2)) on the carbonyl group in the moiety of formula (A2).

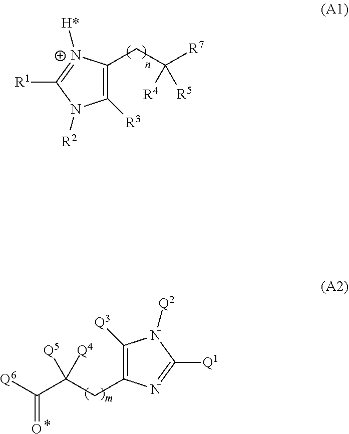

The present application provides a non-covalent dimer cation, comprising or consisting of a cation of formula (A), a tautomer thereof, or a stereoisomer thereof, wherein the bond “-------” in formula (A) is a non-covalent bond.

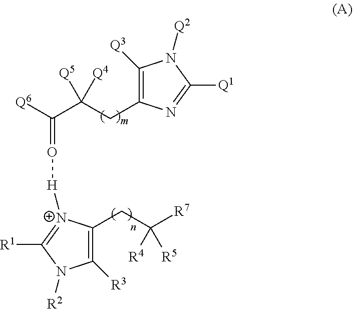

The non-covalent bond, that is, the bond “-------” in formula (A), shall be regarded as comprising or consisting of any of chemically allowed non-covalent bonds that enable the non-covalent dimer cation of the present application to exist stably in its salts.

According to the understanding of those skilled in the art, the non-covalent bond may be understood as a hydrogen bond. However, through experiments, the applicant has confirmed that the non-covalent exists stably in the general environment, for example, in the form of a salt; in particular, the non-covalent bond can remain stable without being broken at least during determination by mass spectroscopy, that is, the non-covalent dimer cation of the present application can be kept intact during determination by mass spectroscopy, and accordingly an ion peak corresponding to an m/z value of the non-covalent dimer cation of the present application is generated. That is, it should be understood that properties of some hydrogen bonds may be applicable to the non-covalent bond, however, the non-covalent bond does not necessarily comply with all understandings of properties of hydrogen bonds by those skilled in the art, in particular in terms of bond energy. In some embodiments, the non-covalent bond is a bond with a bond energy stronger than that of a general NH—:O═C bond, such as a bond with a bond energy greater than 8 kJ/mol.

The cation of formula (A) with the non-covalent bond has stronger reducing capacity compared to a monomer moiety thereof (the moiety of formula (A1) or a moiety of formula (A1) that has not yet been protonated, or the moiety of formula (A2) or a protonated moiety of formula (A2)), which enables it to be a better reductant or an antioxidant, in particular an antioxidant in an organism.

In some embodiments, R⁷ is selected from a group consisting of hydrogen, hydroxymethyl, —C—R⁶, and —C(═O)—R⁶. In some embodiments, R⁷ is hydrogen. In some embodiments, R⁷ is hydroxymethyl. In some embodiments, R⁷ is —C—R⁶. In some embodiments, R⁷ is —C(═O)—R⁶.

In some embodiments, R¹ is selected from a group consisting of hydrogen, hydroxyl, sulfhydryl, amino, methyl, ethyl, and phenyl. In some embodiments, R¹ is selected from a group consisting of hydrogen, hydroxyl, sulfhydryl, amino, and phenyl. In some embodiments, R¹ is hydrogen. In some embodiments, R¹ is hydroxyl. In some embodiments, R¹ is sulfhydryl. In some embodiments, R¹ is amino. In some embodiments, R¹ is methyl. In some embodiments, R¹ is ethyl. In some embodiments, R¹ is phenyl. In some embodiments, Q¹ is selected from a group consisting of hydrogen, hydroxyl, sulfhydryl, amino, methyl, ethyl, and phenyl. In some embodiments, Q¹ is selected from a group consisting of hydrogen, hydroxyl, sulfhydryl, amino, and phenyl. In some embodiments, Q¹ is hydrogen. In some embodiments, Q¹ is hydroxyl. In some embodiments, Q¹ is sulfhydryl. In some embodiments, Q¹ is amino. In some embodiments, Q¹ is methyl. In some embodiments, Q¹ is ethyl. In some embodiments, Q¹ is phenyl.

In some embodiments, R² is selected from a group consisting of hydrogen, methyl, ethyl, propyl, phenyl, benzyloxymethyl, and triphenylmethyl. In some embodiments, R² is selected from a group consisting of hydrogen, methyl, propyl, and triphenylmethyl. In some embodiments, R² is hydrogen. In some embodiments, R² is methyl. In some embodiments, R² is ethyl. In some embodiments, R² is propyl. In some embodiments, R² is phenyl. In some embodiments, R² is benzyloxymethyl. In some embodiments, R² is triphenylmethyl. In some embodiments, Q² is selected from a group consisting of hydrogen, methyl, ethyl, propyl, phenyl, benzyloxymethyl, and triphenylmethyl. In some embodiments, Q² is selected from a group consisting of hydrogen, methyl, propyl, and triphenylmethyl. In some embodiments, Q² is hydrogen. In some embodiments, Q² is methyl. In some embodiments, Q² is ethyl. In some embodiments, Q² is propyl. In some embodiments, Q² is phenyl. In some embodiments, Q² is benzyloxymethyl. In some embodiments, Q² is triphenylmethyl.

In some embodiments, R³ is selected from a group consisting of hydrogen, hydroxyl, sulfhydryl, amino, methyl, ethyl, and phenyl. In some embodiments, R³ is selected from a group consisting of hydrogen, sulfhydryl, and methyl. In some embodiments, R³ is hydrogen. In some embodiments, R³ is hydroxyl. In some embodiments, R³ is sulfhydryl. In some embodiments, R³ is amino. In some embodiments, R³ is methyl. In some embodiments, R³ is ethyl. In some embodiments, R³ is phenyl. In some embodiments, Q³ is selected from a group consisting of hydrogen, hydroxyl, sulfhydryl, amino, methyl, ethyl, and phenyl. In some embodiments, Q³ is selected from a group consisting of hydrogen, sulfhydryl, and methyl. In some embodiments, Q³ is hydrogen. In some embodiments, Q³ is hydroxyl. In some embodiments, Q³ is sulfhydryl. In some embodiments, Q³ is amino. In some embodiments, Q³ is methyl. In some embodiments, Q³ is ethyl. In some embodiments, Q³ is phenyl.

In some embodiments, R⁴ is selected from a group consisting of hydrogen, methyl, ethyl, and propyl. In some embodiments, R⁴ is selected from a group consisting of hydrogen and methyl. In some embodiments, R⁴ is hydrogen. In some embodiments, R⁴ is methyl. In some embodiments, R⁴ is ethyl. In some embodiments, R⁴ is propyl. In some embodiments, Q⁴ is selected from a group consisting of hydrogen, methyl, ethyl, and propyl. In some embodiments, Q⁴ is selected from a group consisting of hydrogen and methyl. In some embodiments, Q⁴ is hydrogen. In some embodiments, Q⁴ is methyl. In some embodiments, Q⁴ is ethyl. In some embodiments, Q⁴ is propyl.

In some embodiments, R⁵ is selected from a group consisting of hydrogen, benzyl, amino, methylamino, hydrazinyl, trimethylammonium, pyrrolyl, and a substituent derived from an amino group further bonded with the C-terminal carbon atom of an amino acid or peptide. In some embodiments, Q⁵ is selected from a group consisting of hydrogen, benzyl, amino, methylamino, hydrazinyl, trimethylammonium, pyrrolyl, and a substituent derived from an amino group further bonded with the C-terminal carbon atom of an amino acid or peptide. In some embodiments, the substituent derived from an amino group further bonded with the C-terminal carbon atom of an amino acid or peptide may be represented as formula “—N—C_{AA}(═O)—Z”, where, C_{AA} is the C-terminal carbon atom of an amino acid or peptide, and Z is the remaining moiety of the amino acid or peptide; that is, formula “Z—C_{AA}OOH” is an amino acid or peptide, in particular an amino acid, dipeptide, or tripeptide. In some embodiments, the substituent derived from an amino group further bonded with the C-terminal carbon atom of an amino acid or peptide is a substituent derived from an amino group further bonded with the C-terminal carbon atom of an amino acid, dipeptide, or tripeptide. In some embodiments, R⁵ is selected from a group consisting of hydrogen, benzyl, amino, methylamino, hydrazinyl, trimethylammonium, pyrrolyl, and a substituent derived from an amino group further bonded with the C-terminal carbon atom of an amino acid, dipeptide, or tripeptide. In some embodiments, Q⁵ is selected from a group consisting of hydrogen, benzyl, amino, methylamino, hydrazinyl, trimethylammonium, pyrrolyl, and a substituent derived from an amino group further bonded with the C-terminal carbon atom of an amino acid, dipeptide, or tripeptide. In some embodiments, the substituent derived from an amino group further bonded with the C-terminal carbon atom of an amino acid or peptide is selected from a group consisting of acetamido, 2-aminoacetamido (i.e. glycylamino, a substituent derived from an amino group further bonded with the C-terminal carbon atom of glycine), 3-aminopropionamido (i.e. β-alanylamino, a substituent derived from an amino group further bonded with the C-terminal carbon atom of β-alanine), and pyroglutamylamino (i.e. a substituent derived from an amino group further bonded with the C-terminal carbon atom of pyroglutamic acid). In some embodiments, R⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino. In some embodiments, R⁵ is selected from a group consisting of hydrogen, amino, hydrazinyl, trimethylammonium, pyrrolyl, acetamido, 2-aminoacetamido, 3-aminopropionamido, and pyroglutamylamino. In some embodiments, R⁵ is hydrogen. In some embodiments, R⁵ is benzyl. In some embodiments, R⁵ is amino. In some embodiments, R⁵ is methylamino. In some embodiments, R⁵ is hydrazinyl. In some embodiments, R⁵ is trimethylammonium. In some embodiments, R⁵ is pyrrolyl. In some embodiments, R⁵ is a substituent derived from an amino group further bonded with the C-terminal carbon atom of an amino acid or peptide. In some embodiments, R⁵ is acetamido. In some embodiments, R⁵ is 2-aminoacetamido. In some embodiments, R⁵ is 3-aminopropionamido. In some embodiments, R⁵ is pyroglutamylamino. In some embodiments, Q⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino. In some embodiments, Q⁵ is selected from a group consisting of hydrogen, amino, hydrazinyl, trimethylammonium, pyrrolyl, acetamido, 2-aminoacetamido, 3-aminopropionamido, and pyroglutamylamino. In some embodiments, Q⁵ is hydrogen. In some embodiments, Q⁵ is benzyl. In some embodiments, Q⁵ is amino. In some embodiments, Q⁵ is methylamino. In some embodiments, Q⁵ is hydrazinyl. In some embodiments, Q⁵ is trimethylammonium. In some embodiments, Q⁵ is pyrrolyl. In some embodiments, Q⁵ is a substituent derived from an amino group further bonded with the C-terminal carbon atom of an amino acid or peptide. In some embodiments, Q⁵ is acetamido. In some embodiments, Q⁵ is 2-aminoacetamido. In some embodiments, Q⁵ is 3-aminopropionamido. In some embodiments, Q⁵ is pyroglutamylamino.

In some embodiments, R⁶ is selected from a group consisting of hydroxyl, oxido, methoxy, and a substituent derived from the N-terminal nitrogen atom of an amino acid or peptide. In some embodiments, Q⁶ is selected from a group consisting of hydroxyl, oxido, methoxy, and a substituent derived from the N-terminal nitrogen atom of an amino acid or peptide. In some embodiments, the substituent derived from the N-terminal nitrogen atom of an amino acid or peptide may be represented as formula “—N_{AA}H—Z”, where, N_{AA} is the N-terminal nitrogen atom of an amino acid or peptide, and Z is the remaining moiety of the amino acid or peptide; that is, formula “Z—N_{AA}H₂” is an amino acid or peptide, in particular is an amino acid, dipeptide, or tripeptide. In some embodiments, the substituent derived from the N-terminal nitrogen atom of an amino acid or peptide is a substituent derived from the N-terminal nitrogen atom of an amino acid, dipeptide, or tripeptide. In some embodiments, R⁶ is selected from a group consisting of hydroxyl, oxido, methoxy, and a substituent derived from the N-terminal nitrogen atom of an amino acid, dipeptide, or tripeptide. In some embodiments, Q⁶ is selected from a group consisting of hydroxyl, oxido, methoxy, and a substituent derived from the N-terminal nitrogen atom of an amino acid, dipeptide, or tripeptide. In some embodiments, the substituent derived from the N-terminal nitrogen atom of an amino acid or peptide is selected from a group consisting of N²-lysino (i.e. a substituent derived from N² nitrogen atom of L-lysine) and glycinoprolino (i.e. a substituent derived from the N-terminal nitrogen atom of L-prolyl-Lglycine). In some embodiments, R⁶ is selected from a group consisting of hydroxyl, oxido, methoxy, N²-lysino, and glycinoprolino. In some embodiments, R⁶ is hydroxyl. In some embodiments, R⁶ is oxido. In some embodiments, R⁶ is methoxy. In some embodiments, R⁶ is a substituent derived from the N-terminal nitrogen atom of an amino acid or peptide. In some embodiments, R⁶ is N²-lysino. In some embodiments, R⁶ is glycinoprolino. In some embodiments, Q⁶ is selected from a group consisting of hydroxyl, oxido, methoxy, N²-lysino, and glycinoprolino. In some embodiments, Q⁶ is hydroxyl. In some embodiments, Q⁶ is oxido. In some embodiments, Q⁶ is methoxy. In some embodiments, Q⁶ is a substituent derived from the N-terminal nitrogen atom of an amino acid or peptide. In some embodiments, Q⁶ is N²-lysino. In some embodiments, Q⁶ is glycinoprolino.

In some embodiments, m and n are each independently selected from a group consisting of 0 and 1. In some embodiments, m is 1. In some embodiments, m is 0. In some embodiments, n is 1. In some embodiments, n is 0. In some embodiments, m is 1, and n is 1. In some embodiments, m is 1, and n is 0. In some embodiments, m is 0, and n is 1. In some embodiments, m is 0, and n is 0.

In some embodiments, R¹ is the same as Q¹. In some embodiments, R² is the same as Q². In some embodiments, R³ is the same as Q³. In some embodiments, R⁴ is the same as Q⁴. In some embodiments, R⁵ is the same as Q⁵. In some embodiments, R⁶ is the same as Q⁶. In some embodiments, R¹ is different from Q¹. In some embodiments, R² is different from Q². In some embodiments, R³ is different from Q³. In some embodiments, R⁴ is different from Q⁴. In some embodiments, R⁵ is different from Q⁵. In some embodiments, R⁶ is different from Q⁶.

In some embodiments: R⁴ is hydrogen; R⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino; R⁶ is selected from a group consisting of hydroxyl, oxido, methoxy, N²-lysino, and glycinoprolino; and n is 1. In some embodiments: Q⁴ is hydrogen; Q⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino; Q⁶ is selected from a group consisting of hydroxyl, oxido, methoxy, N²-lysino, and glycinoprolino; and m is 1. In some embodiments: R⁴ is hydrogen; R⁵ is amino or 3-aminopropionamido; and R⁷ is —C(═O)—R⁶ and R⁶ is hydroxyl, or R⁷ is hydrogen. In some embodiments: R⁴ is hydrogen; R⁵ is amino; R⁷ is —C(═O)—R⁶; and R⁶ is hydroxyl. In some embodiments: R⁴ is hydrogen; R⁵ is 3-aminopropionamido; R⁷ is —C(=O)—R⁶; and R⁶ is hydroxyl. In some embodiments: R⁴ is hydrogen; R⁵ is amino; and R⁷ is hydrogen. In some embodiments: Q⁴ is hydrogen; Q⁵ is amino; and Q⁶ is hydroxyl. In some embodiments: R¹ is hydrogen; R² is hydrogen; and R³ is hydrogen. In some embodiments: Q¹ is hydrogen; Q² is hydrogen; and Q³ is hydrogen. In some embodiments, R⁷ is —C(═O)—R⁶, R¹ is the same as Q¹, R² is the same as Q², R³ is the same as Q³, R⁴ is the same as Q⁴, R⁵ is the same as Q⁵, and the R⁶ is the same as Q⁶. In some embodiments, at least two of R⁴, R⁵, and R⁷ are hydrogen. In some embodiments, R⁴ is hydrogen, and R⁵ is hydrogen. In some embodiments, R⁵ is hydrogen, and R⁷ is hydrogen. In some embodiments, Q⁴ is hydrogen, and Q⁵ is hydrogen.

In the present application, any disclosure or inclusion of a chemical structure specified by formula (A) and/or any selectable specific moiety R¹, R², R³, R⁴, R⁵, R⁶, R⁷, Q¹, Q², Q³, Q⁴, Q⁵, and/or Q⁶ together with the specific coefficients m and n in other structural formulas shall be regarded as a further disclosure or inclusion of a stereoisomer of the specified chemical structure. In the present application, in particular in embodiments, any disclosure or inclusion of a chemical structure shall be regarded as a further disclosure or inclusion of a stereoisomer of the chemical structure.

In the present application, if the stereochemistry of any one or more chiral centers is not specified, it shall be regarded as a disclosure or inclusion of possible stereoisomers of the chiral center or combinations or mixtures of the stereoisomers. For example, if the stereochemistry of a chiral center is not specified, it shall be regarded as a disclosure or inclusion of respectively a chemical structure of the chiral center in the (R)-configuration, a chemical structure of the chiral center in the (S)-configuration, and a combination of the chiral center in any ratio, including a racemic mixture. For example, if the stereochemistry of two chiral centers is not specified, it shall be regarded as respectively disclosure or inclusion of a chemical structure of which two chiral centers are in the (R,R)-configuration, a chemical structure of which two chiral centers are in the (S,S)-configuration, a chemical structure of which two chiral centers are in the (R,S)-configuration, a chemical structure of which two chiral centers are in the (S,R)-configuration, and a combination thereof in any ratio.

In some embodiments, R⁴, R⁵, and R⁷ are different from each other; for example, R⁴ is different from R⁵, and R⁷ is selected from a group consisting of hydroxymethyl and —C(═O)—R⁶, in particular —C(═O)—R⁶. In this case, a carbon to which R⁴, R⁵, and R⁷ are bonded is an asymmetric carbon (hereinafter referred to as a “first asymmetric carbon”).

As a specific example of the foregoing definition, any disclosure or inclusion of a chemical structure comprising the first asymmetric carbon, in a case that a configuration of the first asymmetric carbon is not further indicated, shall be regarded as also a further disclosure or inclusion of two possible configurations of the first asymmetric carbon. For example, any disclosure or inclusion of moiety of formula (A1), in a case that the configuration of the first asymmetric carbon is not further indicated, shall be regarded as also further disclosures or inclusions of moiety of formula (A1-R) and moiety of formula (A1-S). In some embodiments, in formula (A) or formula (A1), a bond between the first asymmetric carbon and R⁴ is a bond of R⁴ that is in front of the plane of the paper, and a bond between the first asymmetric carbon and R⁵ is a bond of R⁵ that is behind the plane of the paper. That is the moiety of formula (A1-R), wherein the first asymmetric carbon is represented by “*”. In a case that the atom, bonded to the first asymmetric carbon, of R⁵ is a nitrogen atom, and R⁷ is selected from a group consisting of hydroxymethyl and —C(═O)—R⁶, the first asymmetric carbon is generally in the R-configuration. D-histidine has this configuration. In some embodiments, in formula (A) or formula (A1), a bond between the first asymmetric carbon and R⁴ is a bond of R⁴ that is behind the plane of the paper, and a bond between the first asymmetric carbon and R⁵ is a bond of R⁵ that is in front of the plane of the paper. That is the moiety of formula (A1-S), wherein the first asymmetric carbon is represented by “*”. In a case that an atom, bonded to the first asymmetric carbon, of R⁵ is a nitrogen atom, and R⁷ is selected from a group consisting of hydroxymethyl and —C(═O)—R⁶, the first asymmetric carbon is generally in the S-configuration. L-histidine has this configuration.

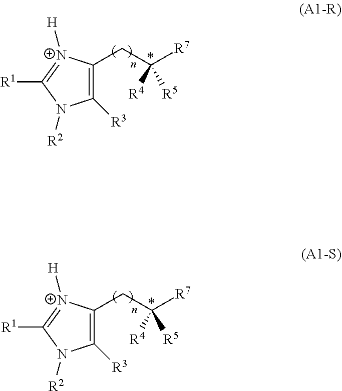

In some embodiments, Q⁴ is different from Q⁵. In this case, a carbon to which Q⁴ and Q⁵ are bonded is an asymmetric carbon (hereinafter referred to as a “second asymmetric carbon”).

As a specific example of the foregoing definition, any disclosure or inclusion of a chemical structure comprising the second asymmetric carbon, in a case that a configuration of the second asymmetric carbon is not further indicated, shall be regarded as also a further disclosure or inclusion of two possible configurations of the second asymmetric carbon. For example, any disclosure or inclusion of moiety of formula (A2), in a case that the configuration of the second asymmetric carbon is not further indicated, shall be regarded as also further disclosures or inclusions of moiety of formula (A2-R) and moiety of formula (A2-S). In some embodiments, in formula (A) or formula (A2), a bond between the second asymmetric carbon and Q⁴ is a bond of Q⁴ that is in front of the plane of the paper, and a bond between the second asymmetric carbon and Q⁵ is a bond of Q⁴ that is behind the plane of the paper. That is the moiety of formula (A2-R), wherein the second asymmetric carbon is represented by “*”. In a case that an atom, bonded to the second asymmetric carbon, of Q⁵ is a nitrogen atom, the second asymmetric carbon is generally in the R-configuration. D-histidine has this configuration. In some embodiments, in formula (A) or formula (A2), a bond between the second asymmetric carbon and Q⁴ is a bond of Q⁴ that is behind the plane of the paper, and a bond between the second asymmetric carbon and Q⁵ is a bond of Q⁵ that is in front of the plane of the paper. That is the moiety of formula (A2-S), wherein the second asymmetric carbon is represented by “*”. In a case that an atom, bonded to the second asymmetric carbon, of Q⁴ is a nitrogen atom, the second asymmetric carbon is generally in the S-configuration. L-histidine has this configuration.

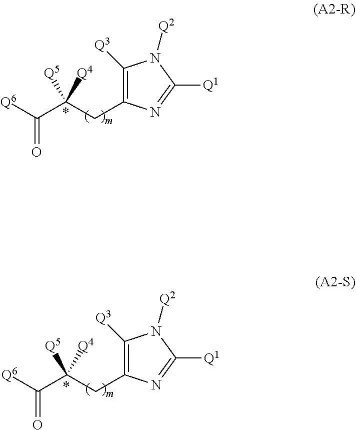

In some embodiments, the first asymmetric carbon is in the (R)-configuration. In some embodiments, the first asymmetric carbon is in the (S)-configuration. In some embodiments, the second asymmetric carbon is in the (R)-configuration. In some embodiments, the second asymmetric carbon is in the (S)-configuration. In some embodiments, the first asymmetric carbon is in the (R)-configuration, and the second asymmetric carbon is in the (R)-configuration. In some embodiments, the first asymmetric carbon is in the (R)-configuration, and the second asymmetric carbon is in the (S)-configuration. In some embodiments, the first asymmetric carbon is in the (S)-configuration, and the second asymmetric carbon is in the (R)-configuration. In some embodiments, the first asymmetric carbon is in the (S)-configuration, and the second asymmetric carbon is in the (S)-configuration.

In some embodiments, in formula (A), a bond between the first asymmetric carbon and R⁴ is a bond of R⁴ that is in front of the plane of the paper, a bond between the first asymmetric carbon and R⁵ is a bond of R⁵ that is behind the plane of the paper, a bond between the second asymmetric carbon and Q⁴ is a bond of Q⁴ that is in front of the plane of the paper, and a bond between the second asymmetric carbon and Q⁵ is a bond of Q⁵ that is behind the plane of the paper. In some embodiments, in formula (A), a bond between the first asymmetric carbon and R⁴ is a bond of R⁴ that is in front of the plane of the paper, a bond between the first asymmetric carbon and R⁵ is a bond of R⁵ that is behind the plane of the paper, a bond between the second asymmetric carbon and Q⁴ is a bond of Q⁴ that is behind the plane of the paper, and a bond between the second asymmetric carbon and Q⁵ is a bond of Q⁵ that is in front of the plane of the paper. In some embodiments, in formula (A), a bond between the first asymmetric carbon and R⁴ is a bond of R⁴ that is behind the plane of the paper, a bond between the first asymmetric carbon and R⁵ is a bond of R⁵ that is in front of the plane of the paper, a bond between the second asymmetric carbon and Q⁴ is a bond of Q⁴ that is in front of the plane of the paper, and a bond between the second asymmetric carbon and Q⁵ is a bond of Q⁵ that is behind the plane of the paper. In some embodiments, in formula (A), a bond between the first asymmetric carbon and R⁴ is a bond of R⁴ that is behind the plane of the paper, a bond between the first asymmetric carbon and R⁵ is a bond of R⁵ that is in front of the plane of the paper, a bond between the second asymmetric carbon and Q⁴ is a bond of Q⁴ that is behind the plane of the paper, and a bond between the second asymmetric carbon and Q⁵ is a bond of Q⁵ that is in front of the plane of the paper.

In some embodiments, R⁴ is different from R⁵, R⁷ is selected from a group consisting of hydroxymethyl and —C(═O)—R⁶, and Q⁴ is different from Q⁵. In some embodiments, R⁴ is different from R⁵, R⁷ is selected from a group consisting of hydroxymethyl and —C(═O)—R⁶, Q⁴ is different from Q⁵, the first asymmetric carbon is in the (R)-configuration, and the second asymmetric carbon is in the (R)-configuration; as shown in formula (A-RR). In some embodiments, R⁴ is different from R⁵, R⁷ is selected from a group consisting of hydroxymethyl and —C(═O)—R⁶, Q⁴ is different from Q⁵, the first asymmetric carbon is in the (R)-configuration, and the second asymmetric carbon is in the (S)-configuration; as shown in formula (A-RS). In some embodiments, R⁴ is different from R⁵, R⁷ is selected from a group consisting of hydroxymethyl and —C(═O)—R⁶, Q⁴ is different from Q⁵, the first asymmetric carbon is in the (S)-configuration, and the second asymmetric carbon is in the (R)-configuration; as shown in formula (A-SR). In some embodiments, R⁴ is different from R⁵, R⁷ is selected from a group consisting of hydroxymethyl and —C(═O)—R⁶, Q⁴ is different from Q⁵, the first asymmetric carbon is in the (S)-configuration, and the second asymmetric carbon is in the (S)-configuration; as shown in formula (A-SS).

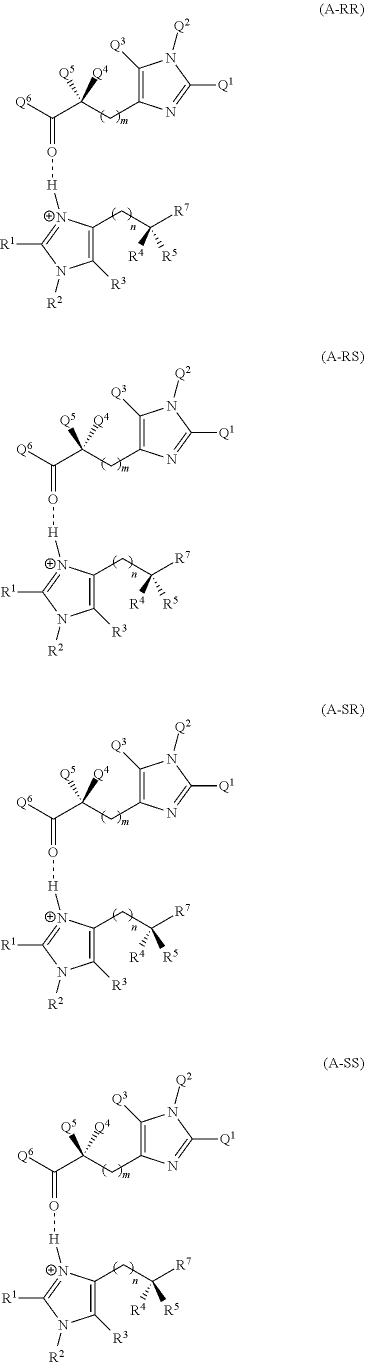

In the present application, any disclosure or inclusion of a chemical structure specified by formula (A) and/or in other structural formulas with any selection of the specific moiety R¹, R², R³, R⁴, R⁵, R⁶, R⁷, Q¹, Q², Q³, Q⁴, Q⁵, and/or Q⁶ as well as the specific coefficients m and n, shall be regarded as a further disclosure or inclusion of a chemically allowed tautomer of the specified chemical structure. In the present application, in particular in embodiments, any disclosure or inclusion of a chemical structure shall be regarded as a further disclosure or inclusion of a chemically allowed tautomer, such as a keto-enol tautomer, of the chemical structure.

As a specific example of the foregoing definition, any disclosure or inclusion of a chemical structure of a moiety comprising “a carbon on a double bond or on an aromatic ring, as well as hydroxyl or sulfhydryl thereon”, shall be regarded as also a further disclosure or inclusion of a corresponding tautomer, that is, a chemical structure comprising a “carbonyl or thiocarbonyl” moiety. For example, any disclosure or inclusion of a chemical structure comprising a

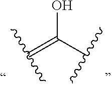 [SMILES: *C(=*)O]

moiety shall be regarded as a further disclosure or inclusion of a corresponding tautomer comprising a

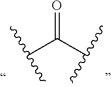 [SMILES: *C(*)=O]

moiety; any disclosure or inclusion of a chemical structure comprising a

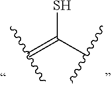 [SMILES: *C(=*)S]

moiety shall be regarded as a further disclosure or inclusion of a corresponding tautomer comprising a

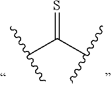 [SMILES: *C(*)=S]

moiety.

As a specific example of the foregoing definition, any disclosure or inclusion of a chemical structure comprising a

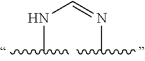 [SMILES: */N=C\N*]

moiety in an aromatic ring shall be regarded as also a further disclosure or inclusion of a corresponding tautomer comprising a

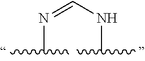 [SMILES: */N=C\N*]

moiety. For example, any disclosure or inclusion of a chemical structure comprising an imidazole ring

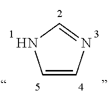

moiety shall be regarded as a further disclosure or inclusion of a corresponding tautomer comprising a

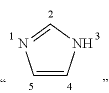

moiety.

In the present application, any disclosure or inclusion of a resonance structure constituting a hybrid shall be regarded as a further disclosure or inclusion of all other resonance structure constituting the hybrid as well as the hybrid itself.

As a specific example of the foregoing definition, any disclosure or inclusion of a chemical structure comprising a

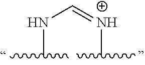 [SMILES: *N/C=[NH+]\*]

moiety in an aromatic ring shall be regarded as also a further disclosure or inclusion of a corresponding resonance chemical structure comprising a

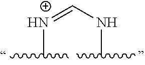 [SMILES: *N/C=[NH+]\*]

moiety. For example, any disclosure or inclusion of a chemical structure comprising an imidazole ring

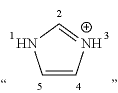

moiety shall be regarded as a further disclosure or inclusion of a resonance chemical structure comprising a

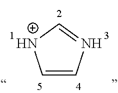

moiety, and more a further disclosure or inclusion of a corresponding hybrid (for example, represented as

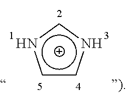

In the present application, any disclosure or inclusion of a chemical structure comprising a non-covalent bond, such as a hydrogen bond, that is formed by hydrogen on N¹ in a protonated imidazole ring

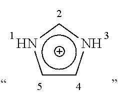

shall be regarded as also a further disclosure or inclusion of a corresponding chemical structure comprising a non-covalent bond, such as a hydrogen bond, that is formed by hydrogen on N³ in a protonated imidazole ring

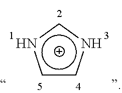

In the present application, any disclosure or inclusion of a chemical structure comprising a non-covalent bond, such as a hydrogen bond, that is formed by hydrogen on N^{τ} in protonated histidine, protonated histamine, protonated sarcosine, or a similar compound with 1H-imidazole-4-yl or a group derived from 1H-imidazole-4-yl, shall be regarded as also a further disclosure or inclusion of a corresponding chemical structure comprising a non-covalent bond, such as a hydrogen bond, that is formed by hydrogen on N^{π} in protonated histidine, protonated histamine, protonated sarcosine, or a similar compound with 1H-imidazole-4-yl or a group derived from 1H-imidazole-4-yl.

In the present application, any disclosure or inclusion of a chemical structure in which R² is hydrogen, shall be regarded as not only a disclosure or inclusion of a chemical structure in which a non-covalent bond is formed by hydrogen (N^{π}-H) that forms a non-covalent bond as shown as formula (A), but also a further disclosure or inclusion of a corresponding chemical structure in which a non-covalent bond is formed by hydrogen (A^{τ}-H) on R², and vice versa. It should be understood that a structure of formula (A) in which R² is hydrogen and its corresponding structure of formula (A′) may be tautomers. In the present application, any disclosure or inclusion of a structure of formula (A) in which R² is hydrogen, shall be regarded as also a further disclosure or inclusion of a structure of formula (A′), and vice versa.

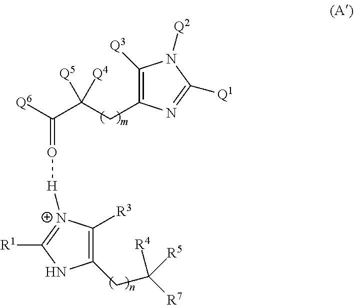

Similar to the structure of formula (A), correspondingly, the structure of formula (A′) also comprises the first asymmetric carbon and the second asymmetric carbon.

In some embodiments, the structure of formula (A′) is shown as formula (A′-RR). In some embodiments, the structure of formula (A′) is shown as formula (A′-RS). In some embodiments, the structure of formula (A′) is shown as formula (A′-SR). In some embodiments, the structure of formula (A′) is shown as formula (A′-SS).

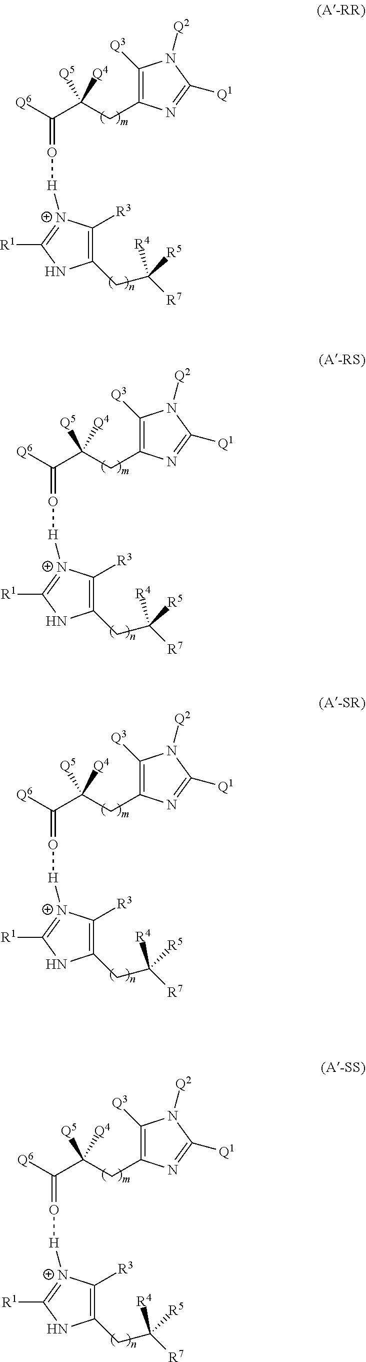

In the present application, any disclosure or inclusion of any one of the (Brønsted-Lowry) conjugate acid or base, shall be regarded as a further disclosure or inclusion of the corresponding conjugate acids and bases existing or mainly existing under certain pH conditions that is chemically reasonable and that does not affect the main chemical structure and technical effects of the non-covalent dimer cation or salt of the present application. For example, under the condition of chemically reasonable and not affecting the main chemical structure and technical effects of the non-covalent dimer cation or salt of the present application, any disclosure or inclusion of specific moiety or group that is a hydroxyl shall be regarded as a further disclosure or inclusion of a specific moiety or group that is oxido group (—O⁻) or oxonio group (—OH₂⁺).

In some embodiments, R¹ is hydrogen, R² is hydrogen, R³ is hydrogen, R⁴ is hydrogen, R⁵ is amino, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is amino, R⁷ is hydrogen, and n=1. In some embodiments, R¹ is hydroxyl, R² is hydrogen, R³ is hydrogen, R⁴ is hydrogen, R⁵ is amino, R⁶ is hydroxyl, R⁷ is —C(═O)—R⁶, and n=1. In some embodiments, R¹ is sulfhydryl, R² is hydrogen, R³ is hydrogen, R⁴ is hydrogen, R⁵ is amino, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is 3-aminopropionamido, R⁶ is hydroxyl, R⁷ is —C(═O)—R⁶, and n=1. In some embodiments, R¹ is hydrogen, R² is triphenylmethyl, R³ is hydrogen, R⁴ is hydrogen, R⁵ is amino, R⁶ is methoxy, R⁷ is —C(═O)—R⁶, and n=1. In some embodiments, R¹ is sulfhydryl, R² is hydrogen, R³ is hydrogen, R⁴ is hydrogen, R⁵ is trimethylammonium, R⁶ is oxido, R⁷ is —C(═O)—R⁶, and n=1. In some embodiments, R¹ is hydrogen, R² is hydrogen, R³ is methyl, R⁴ is hydrogen, R⁵ is hydrogen, R⁶ is hydroxyl, R⁷ is —C(═O)—R⁶, and n=0. In some embodiments, R¹ is hydrogen, R² is hydrogen, R³ is hydrogen, R⁴ is hydrogen, R⁵ is 2-aminoacetamido, R⁶ is N² -lysino, R⁷ is —C(═O)—R⁶, and n=1. In some embodiments, R¹ is hydrogen, R² is hydrogen, R³ is hydrogen, R⁴ is hydrogen, R⁵ is pyroglutamylamino, R⁶ is glycinoprolino, R⁷ is —C(═O)—R⁶, and n=1. In some embodiments, R¹ is hydrogen, R² is hydrogen, R³ is hydrogen, R⁴ is amino, R⁵ is methyl, R⁶ is hydroxyl, R⁷ is —C(═O)—R⁶, and n=1. In some embodiments, R¹ is amino, R² is hydrogen, R³ is hydrogen, R⁴ is hydrogen, R⁵ is amino, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is amino, R⁶ is methoxy, R⁷ is —C(═O)—R⁶, and n=1. In some embodiments, R¹ is hydrogen, R² is hydrogen, R³ is hydrogen, R⁴ is hydrogen, R⁵ is hydrazino, R⁶ is hydroxyl, R⁷ is —C(═O)—R⁶, and n=1. In some embodiments, R¹ is hydrogen, R² is methyl, R³ is hydrogen, R⁴ is hydrogen, R⁵ is amino, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is acetamido, R⁶ is hydroxyl, R⁷ is —C(═O)—R⁶, and n=1. In some embodiments, R¹ is hydrogen, R² is hydrogen, R³ is sulfhydryl, R⁴ is hydrogen, R⁵ is amino, R⁶ is hydroxyl, R⁷ is —C(═O)—R⁶, and n=1. In some embodiments, R¹ is phenyl, R² is hydrogen, R³ is hydrogen, R⁴ is hydrogen, R⁵ is amino, R⁶ is hydroxyl, R⁷ is —C(═O)—R⁶, and n=1. In some embodiments, R¹ is hydrogen, R² is propyl, R³ is hydrogen, R⁴ is hydrogen, R⁵ is amino, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is pyrrolyl, R⁶ is hydroxyl, R⁷ is —C(═O)—R⁶, and n=1. In some embodiments, R¹ is hydrogen, R² is hydrogen, R³ is hydrogen, R⁴ is hydrogen, R⁵ is amino, R⁷ is hydroxymethyl, and n=1.

In some embodiments, Q¹ is hydrogen, Q² is hydrogen, Q³ is hydrogen, Q⁴ is hydrogen, Q⁵ is amino, Q⁶ is hydroxyl, and m=1. In some embodiments, Q¹ is hydroxyl, Q² is hydrogen, Q³ is hydrogen, Q⁴ is hydrogen, Q⁵ is amino, Q⁶ is hydroxyl, and m=1. In some embodiments, Q¹ is sulfhydryl, Q² is hydrogen, Q³ is hydrogen, Q⁴ is hydrogen, Q⁵ is amino, Q⁶ is hydroxyl, and m=1. In some embodiments, Q¹ is sulfhydryl, Q² is hydrogen, Q³ is hydrogen, Q⁴ is hydrogen, Q⁵ is trimethylammonium, Q⁶ is oxido, and m=1. In some embodiments, Q¹ is hydrogen, Q² is triphenylmethyl, Q³ is hydrogen, Q⁴ is hydrogen, Q⁴ is amino, Q⁶ is methoxy, and m=1. In some embodiments, Q¹ is hydrogen, Q² is hydrogen, Q³ is methyl, Q⁴ is hydrogen, CQ⁵ is hydrogen, Q⁶ is hydroxyl, and m=0. In some embodiments, Q¹ is hydrogen, Q² is hydrogen, Q³ is hydrogen, Q⁴ is hydrogen, Q⁵ is 2-aminoacetamido, Q⁶ is N²-lysino, and m=1. In some embodiments, Q¹ is hydrogen, Q² is hydrogen, Q³ is hydrogen, Q⁴ is hydrogen, Q⁵ is pyroglutamylamino, Q⁶ is glycinoprolino, and m=1. In some embodiments, Q¹ is hydrogen, Q² is hydrogen, Q³ is hydrogen, Q⁴ is amino, Q⁵ is methyl, Q⁶ is hydroxyl, and m=1. In some embodiments, Q¹ is amino, Q² is hydrogen, Q³ is hydrogen, Q⁴ is hydrogen, Q⁵ is amino, Q⁶ is hydroxyl, and m=1. In some embodiments, Q¹ is hydrogen, Q² is hydrogen, Q³ is hydrogen, Q⁴ is hydrogen, Q⁵ is amino, Q⁶ is methoxy, and m=1. In some embodiments, Q¹ is hydrogen, Q² is hydrogen, Q³ is hydrogen, Q⁴ is hydrogen, Q⁵ is hydrazino, Q⁶ is hydroxyl, and m=1. In some embodiments, Q¹ is hydrogen, Q² is methyl, Q³ is hydrogen, Q⁴ is hydrogen, Q⁵ is amino, Q⁶ is hydroxyl, and m=1. In some embodiments, Q¹ is hydrogen, Q² is hydrogen, Q³ is hydrogen, Q⁴ is hydrogen, Q⁵ is acetamido, Q⁶ is hydroxyl, and m=1. In some embodiments, Q¹ is hydrogen, Q² is hydrogen, Q³ is sulfhydryl, Q⁴ is hydrogen, Q⁵ is amino, Q⁶ is hydroxyl, and m=1. In some embodiments, Q¹ is phenyl, Q² is hydrogen, Q³ is hydrogen, Q⁴ is hydrogen, Q⁵ is amino, Q⁶ is hydroxyl, and m=1. In some embodiments, Q¹ is hydrogen, Q² is hydrogen, Q³ is hydrogen, Q⁴ is hydrogen, Q⁵ is 3-aminopropionamido, Q⁶ is hydroxyl, and m=1. In some embodiments, Q¹ is hydrogen, Q² is propyl, Q³ is hydrogen, Q⁴ is hydrogen, Q⁵ is amino, Q⁵ is hydroxyl, and m=1. In some embodiments, Q¹ is hydrogen, Q² is hydrogen, Q³ is hydrogen, Q⁴ is hydrogen, Q⁵ is pyrrolyl, Q⁶ is hydroxyl, and m=1.

In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is methyl, Q⁴ is hydrogen, Q⁵ is hydrogen, Q⁶ is hydroxyl, n=1, and m=0. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is amino, Q⁶ is hydroxyl, n=1, and m =1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is trimethylammonium, Q⁶ is oxido, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is triphenylmethyl, Q³ is hydrogen, Q⁴ is hydrogen, Q⁵ is amino, Q⁶ is methoxy, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is methyl, Q⁴ is hydrogen, Q⁵ is hydrogen, Q⁶ is hydroxyl, n=1, and m=0. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 2-aminoacetamido, Q⁶ is N² -lysino, n=1, and m=0. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is pyroglutamylamino, Q⁶ is glycinoprolino, n=1, and m=1. In some embodiments, the cation of the present application is shown as formula (A), where, R¹ is hydrogen, R² is hydrogen, R³ is hydrogen, R⁴ is hydrogen, R⁵ is amino, R⁷ is hydrogen, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydroxyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is sulfhydryl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is 3-aminopropionamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is triphenylmethyl, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is sulfhydryl, R² is hydrogen, R³ is hydrogen, R⁴ is hydrogen, R⁵ is trimethylammonium, R⁶ is oxido, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is methyl, R⁴ is hydrogen, R⁵ is hydrogen, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=0, and m=1. In some embodiments, the cation of the present application is shown as formula (A), where, R¹ is hydrogen, R² is hydrogen, R³ is hydrogen, R⁴ is hydrogen, R⁵ is 2-aminoacetamido, R⁶ is N² -lysino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ hydrogen, R² is hydrogen, R³ is hydrogen, R⁴ is hydrogen, R⁵ is pyroglutamylamino, R⁶ is glycinoprolino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is amino, Q⁵ is methyl, Q⁶ is hydroxyl, n=1, and m =1. In some embodiments, the cation of the present application is shown as formula (A), where, R¹ is hydrogen, R² is hydrogen, R³ is hydrogen, R⁴ is amino, R⁵ is methyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is amino, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is amino, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is methoxy, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m =1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is hydrazino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is hydrazino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is methyl, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is methyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is acetamido, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is acetamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is sulfhydryl, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is sulfhydryl, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is phenyl, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is phenyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 3-aminopropionamido, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is propyl, Q¹ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is propyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is pyrrolyl, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is pyrrolyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m=1. In some embodiments, the cation of the present application is shown as formula (A), where, R¹ is hydrogen, R² is hydrogen, R³ is hydrogen, R⁴ is hydrogen, R⁵ is amino, R⁷ is hydroxymethyl, Q¹ is hydrogen, Q² is hydrogen, Q³ is hydrogen, Q⁴ is hydrogen, Q⁵ is amino, Q⁶ is hydroxyl, n=1, and m=1.

### The Salt of the Present Application

In an aspect, the present application provides a salt.

In some embodiments, the salt of the present application comprises the non-covalent dimer cation of the present application, in particular the non-covalent dimer cation of the present application and the first anion. For example, the salt of the present application may be represented by a structure of formula (B) or a structure of formula (B′), wherein X is the first anion.

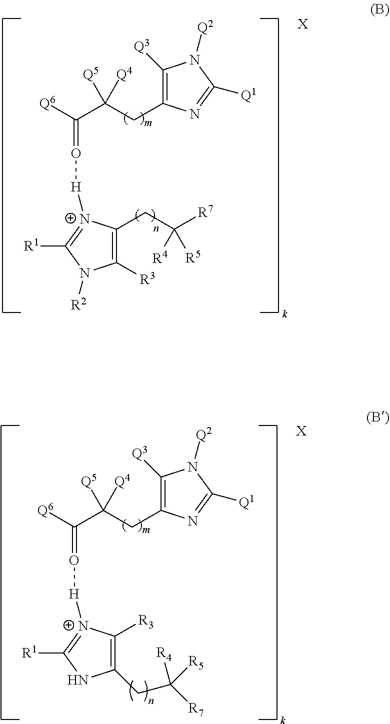

Through the first anion of the present application, the cation of the present application is enabled to exist stably in the form of a salt.

In some embodiments, the first anion (X in formula (B)) comprises or consists of at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion. In some embodiments, the first anion comprises or consists of at least one selected from a group consisting of a hexafluorophosphate ion, a hexafluorosilicate ion, a chloride ion, and a sulfate ion. In some embodiments, the first anion is a fluoride ion. In some embodiments, the first anion is a chloride ion. In some embodiments, the first anion is a bromide ion. In some embodiments, the first anion is an iodide ion. In some embodiments, the first anion is a sulfide ion. In some embodiments, the first anion is a nitrate ion. In some embodiments, the first anion is a sulfate ion. In some embodiments, the first anion is a sulfite ion. In some embodiments, the first anion is a thiosulfate ion. In some embodiments, the first anion is a persulfate ion. In some embodiments, the first anion is a selenate ion. In some embodiments, the first anion is a phosphate ion. In some embodiments, the first anion is a carbonate ion. In some embodiments, the first anion is a hexafluorophosphate ion. In some embodiments, the first anion is a hexafluorosilicate ion. In some embodiments, the first anion is an acetate ion. In some embodiments, the first anion is a sulfonate ion such as a sulfamate ion. In some embodiments, the first anion is a benzoate ion. In some embodiments, the first anion is a polyphosphate ion.

A value of a coefficient k is obtained based on the valence of the non-covalent dimer cation of the present application and the first anion. In a case of the non-covalent dimer cation of the present application with a monovalent positive electron, the value of the coefficient k is equal to the valence of the first anion. In some embodiments, k is 1. In some embodiments, k is 2. In some embodiments, k is 3.

In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=2, and X is hexafluorosilicate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is chloride ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is chloride ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 3-aminopropionamido, Q⁶ is hydroxyl, n=1, m=1, k=2, and X is sulfate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydroxyl, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is trimethylammonium, Q⁶ is oxido, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is triphenylmethyl, Q³ is hydrogen, Q⁴ is hydrogen, Q⁵ is amino, Q⁶ is methoxy, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is methyl, Q⁴ is hydrogen, Q⁵ is hydrogen, Q⁶ is hydroxyl, n=1, m=0, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 2-aminoacetamido, Q⁶ is N² -lysino, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is pyroglutamylamino, Q⁶ is glycinoprolino, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁷ is hydrogen, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydroxyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is sulfhydryl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is 3-aminopropionamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is triphenylmethyl, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is sulfhydryl, R² is hydrogen, R³ is hydrogen, R⁴ is hydrogen, R⁵ is trimethylammonium, R⁶ is oxido, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is methyl, R⁴ is hydrogen, R⁵ is hydrogen, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=0, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is 2-aminoacetamido, R⁶ is N²-lysino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is pyroglutamylamino, R⁶ is glycinoprolino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is amino, Q⁵ is methyl, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is amino, R⁵ is methyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is amino, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is amino, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k =1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Qis methoxy, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is hydrazino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is hydrazino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is methyl, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is methyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is acetamido, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is acetamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R7 is —C(═O)—R⁶, Q¹ is hydrogen, Q² is hydrogen, Q³ is sulfhydryl, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is sulfhydryl, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is phenyl, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is phenyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 3-aminopropionamido, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is propyl, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is propyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is pyrrolyl, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is pyrrolyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion. In some embodiments, the salt of the present application is shown as formula (B), where, R¹ is hydrogen, R² is hydrogen, R³ is hydrogen, R⁴ is hydrogen, R⁵ is amino, R⁷ is hydroxymethyl, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

### Preparation Method of the Non-Covalent Dimer Cation of the Present Application or the Salt of the Present Application

In some embodiments, a preparation method of a salt of the non-covalent dimer cation of the present application, that is, the salt of the present application, is provided, which may comprise the following steps.

### Salt Formation of a First Reactant

A first reactant is contacted with a first acid.

Then, a first acid salt of the first reactant is obtained.

In some embodiments, the first acid salt of the first reactant comprises or consists of the first reactant and the first acid, and/or a conjugate base (a first acid conjugate base) of a protonated first reactant and the first acid.

In some embodiments, the first reactant comprises, or essentially consists of, in particular consists of, a compound of formula (C). In some embodiments, the first acid salt of the first reactant comprises, or essentially consists of, in particular consists of, a salt of formula (C1), wherein A is a first acid conjugate base, a is the valence of the first acid conjugate base, and p is the coefficient of HₐA. R¹,

R², R³, R⁴, R⁵, R⁶, R⁷, and n in formula (C) or formula (C1) may coincide with corresponding moieties or coefficients of a desired cation (formula (A)) or salt (formula (B)) according to embodiments of the present application.

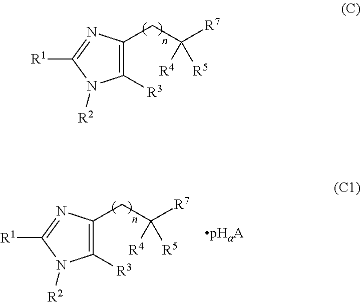

In some embodiments, the first reactant is contacted with the first acid under the presence of a first solvent, and in particular, in the first solvent. In some embodiments, the first acid is at least one selected from a group consisting of nitric acid, phosphoric acid, sulfuric acid, hydrofluoric acid, hydrochloric acid, hydrobromic acid, and hydroiodic acid, in particular hydrochloric acid; that is, the first acid conjugate base (A in formula (C1)) is at least one selected from a group consisting of a nitrate ion, a phosphate ion, a hydrogen phosphate ion, a dihydrogen phosphate ion, a sulfate ion, a hydrogen sulfate ion, a fluoride ion, a chloride ion, a bromide ion, and an iodide ion, in particular a chloride ion. In some embodiments, the first solvent is water. In formula (C1), the coefficients p and a are obtained based on the type of the first acid. For example, in some embodiments, the first acid is hydrochloric acid, and p is 1.

Unless otherwise further defined, the term “conjugate acid” herein is intended to comprise a Brønsted-Lowry conjugate acid of a compound to which the term is directed; furthermore, it does not only include a first conjugate acid obtained in a case that the compound acquires a proton, but also include, if chemically allowed, an acid obtained in a case that the compound further acquires more protons, such as a second conjugate acid obtained in a case that the first conjugate further acquires a proton, a third conjugate acid obtained in a case that the second conjugate acid much further acquires a proton, etc. Unless otherwise further defined, the term “conjugate base” herein is intended to comprise a Brønsted-Lowry conjugate base of a compound to which the term is directed; furthermore, it does not only include a first conjugate base obtained in a case that the compound loses a proton, but also include, if chemically allowed, a base obtained in a case that the compound further loses more protons, such as a second conjugate base obtained in a case that the first conjugate base further loses a proton, a third conjugate base obtained in a case that the second conjugate base much further loses a proton, etc. For example, unless otherwise further defined, a conjugate base of sulfuric acid herein shall be regarded as including not only a hydrogen sulfate ion, but also a sulfate ion. For example, unless otherwise further defined, a conjugate base of phosphoric acid herein shall be regarded as including not only a monohydrogen phosphate ion, but also a bihydrogen phosphate ion and a phosphate ion.

In some embodiments, the first acid is in excess relative to the first reactant. In some embodiments, a molar ratio of the first reactant to the first acid is 1:2 to 1:10, particularly 1:2.5 to 1:8, more particularly 1:4 to 1:6, and more particularly about 1:5.

In some embodiments, a concentration of the first acid in the first solvent is 1 M or more. In some embodiments, the concentration of the first acid in the first solvent is 1 M to 5 M. In some embodiments, the concentration of the first acid in the first solvent is about 2 M.

In some embodiments, the first reactant is contacted with the first acid at a first temperature. In some embodiments, the first temperature is 0° C. to 70° C. In some embodiments, the first temperature is 10° C. to 40° C., more particularly 20° C. to 30° C., and more particularly about 25° C. In some embodiments, the first temperature is 20° C. to 50° C., more particularly 30° C. to 40° C., and more particularly about 37° C. In some embodiments, the first temperature is 0° C. to 20° C., more particularly 0° C. to 10° C., and more particularly about 4° C. In some embodiments, the first temperature is 40° C. to 70° C., more particularly 50° C. to 60° C., and more particularly about 60° C.

In some embodiments, after the first reactant is contacted with the first acid, further performing heating and recrystallization to obtain the first acid salt of the first reactant. In some embodiments, heating temperature is 70° C. to 100° C., particularly 80° C. to 90° C., and more particularly about 85° C.

### Protonation of the First Acid Salt of the First Reactant

In a first acidic environment, contacting the first acid salt of the first reactant with a first salt that comprises a first anion, and in particular, consists of a first cation and the first anion. Then, a first solution that comprises a protonate cation of the first acid salt of the first reactant and a first anion, as shown in formula (C2), is obtained.

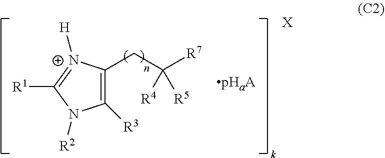

X and k in formula (C2) may coincide with corresponding moieties or coefficients of a desired salt (formula (B)) according to an embodiment of the present application.

In some embodiments, the first salt comprises or consists of at least one selected from a group consisting of ammonium fluoride, ammonium chloride, ammonium bromide, ammonium iodide, ammonium sulfide, ammonium nitrate, ammonium sulfate, ammonium sulfite, ammonium thiosulfate, ammonium persulfate, ammonium selenate, ammonium phosphate, ammonium carbonate, ammonium hexafluorophosphate, ammonium hexafluorosilicate, ammonium acetate, any ammonium sulfonate, ammonium benzoate, and ammonium polyphosphate. In some embodiments, the first salt comprises or consists of at least one selected from a group consisting of ammonium hexafluorophosphate, ammonium hexafluorosilicate, ammonium chloride, and ammonium sulfate. In some embodiments, the first salt is ammonium fluoride. In some embodiments, the first salt is ammonium chloride. In some embodiments, the first salt is ammonium bromide. In some embodiments, the first salt is ammonium iodide. In some embodiments, the first salt is ammonium sulfide. In some embodiments, the first salt is ammonium nitrate. In some embodiments, the first salt is ammonium sulfate. In some embodiments, the first salt is ammonium sulfite. In some embodiments, the first salt is ammonium thiosulfate. In some embodiments, the first salt is ammonium persulfate. In some embodiments, the first salt is ammonium selenate. In some embodiments, the first salt is ammonium phosphate. In some embodiments, the first salt is ammonium carbonate. In some embodiments, the first salt is ammonium hexafluorophosphate. In some embodiments, the first salt is ammonium hexafluorosilicate. In some embodiments, the first salt is ammonium acetate. In some embodiments, the first salt is any ammonium sulfonate such as ammonium sulfamate. In some embodiments, the first salt is ammonium benzoate. In some embodiments, the first salt is ammonium polyphosphate.

In some embodiments, the first acid salt of the first reactant is contacted with the first salt under the presence of a second solvent, and in particular, in the second solvent. In some embodiments, the second solvent is water. In some embodiments, the first cation is an ammonium ion. Here, the ammonium ion, as the first cation, may be taken as a hydrogen donor.

In some embodiments, the first salt is in excess relative to the first acid salt of the first reactant. In some embodiments, a molar ratio of the first acid salt of the first reactant to the first salt is 1:2 to 1:10, particularly 1:2.5 to 1:8, more particularly 1:4 to 1:6, and more particularly about 1:5.

In some embodiments, the first solution further comprises a conjugate base of the first acid. In some embodiments, the first solution further comprises the first cation. In some embodiments, the first solution further comprises the second solvent. In some embodiments, the first solution essentially consists of, in particular consists of the protonate cation of the first acid salt of the first reactant, the first cation, the first anion, the conjugate base of the first acid, and the second solvent.

In some embodiments, the first acid salt of the first reactant is contacted with the first salt at a second temperature. In some embodiments, the second temperature is 0° C. to 70° C. In some embodiments, the second temperature is 10° C. to 40° C., more particularly 20° C. to 30° C., and more particularly about 25° C. In some embodiments, the second temperature is 20° C. to 50° C., more particularly 30° C. to 40° C., and more particularly about 37° C. In some embodiments, the second temperature is 0° C. to 20° C., more particularly 0° C. to 10° C., and more particularly about 4° C. In some embodiments, the second temperature is 40° C. to 70° C., more particularly 50° C. to 60° C., and more particularly about 60° C. In some embodiments, the first temperature is the same as the second temperature.

In some embodiments, the pH of the first acidic environment is less than or equal to 6. In some embodiments, the pH of the first acidic environment is 4 to 6, particularly 4.5 to 5.5, particularly 4.8 to 5.2, particularly 4.9 to 5.1, and more particularly about 5. In general, for the first anion that is less acidic or more basic, a first acidic environment with lower pH is selected. In some embodiments, the first anion is a hexafluorophosphate ion, and the pH of the first acidic environment is 4 to 6, particularly 4.5 to 5.5, particularly 4.8 to 5.2, particularly 4.9 to 5.1, and more particularly about 5.

In some embodiments, the acidic environment is obtained by the first salt in the second solvent. In some embodiments, the first acidic environment is obtained by the first salt and an acid consisting of of the first anion and a hydrogen ion in the second solvent.

### Formation of the Non-Covalent Bond

In a second acidic environment, adding a second reactant to the first solution, and in particular, contacting with the protonated first reactant cation in the first solution. Then, a bond is formed, and a second solution comprising a cation of the first acid salt of the non-covalent dimer cation of the present application and the first anion, as shown in formula (B1) or formula (B1′), is obtained.

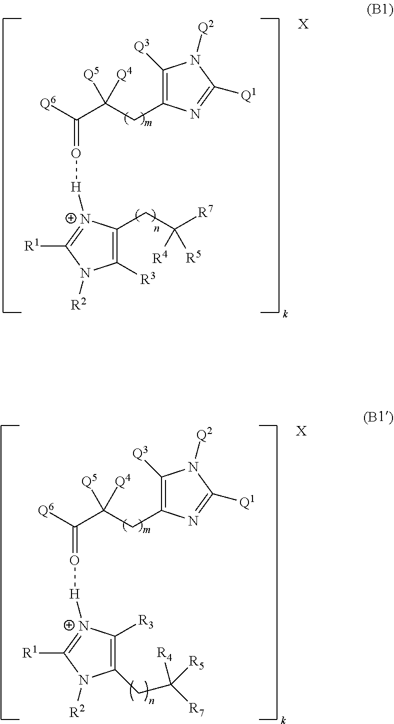

In some embodiments, the second reactant comprises or essentially consists of, in particular consists of, a compound of formula (D), wherein Q¹, Q², Q³, Q⁴, Q⁵, Q⁶, and m may coincide with the desired cation (formula (A)) or salt (formula (B)) according to the embodiments of the present application.

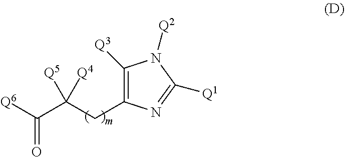

In some embodiments, the second reactant is in excess relative to the protonated first reactant cation. In some embodiments, a molar ratio of the protonated first reactant cation to the second reactant is 1:1 to 1:2, such as about 1:1.0, about 1:1.1, about 1:1.2, about 1:1.3, about 1:1.4, about 1:1.5, about 1:1.6, about 1:1.7, about 1:1.8, about 1:1.9, or about 1:2.0.

In some embodiments, the second solution further comprises a conjugate base of the first acid. In some embodiments, the second solution further comprises the first cation. In some embodiments, the second solution further comprises the second solvent. In some embodiments, the second solution essentially consists of, in particular consists of a cation of the first acid salt of the non-covalent dimer cation of the present application, the first cation, the first anion, the conjugate base of the first acid, and the second solvent.

In some embodiments, the second reactant is added to the first solution at a third temperature. In some embodiments, the third temperature is 0° C. to 70° C. In some embodiments, the third temperature is 10° C. to 40° C., more particularly 20° C. to 30° C., and more particularly about 25° C. In some embodiments, the third temperature is 20° C. to 50° C., more particularly 30° C. to 40° C., and more particularly about 37° C. In some embodiments, the third temperature is 0° C. to 20° C., more particularly 0° C. to 10° C., and more particularly about 4° C. In some embodiments, the third temperature is 40° C. to 70° C., more particularly 50° C. to 60° C., and more particularly about 60° C. In some embodiments, the first temperature is the same as the third temperature. In some embodiments, the second temperature is the same as the third temperature. In some embodiments, the first temperature, the second temperature, and the third temperature are the same.

In some embodiments, the pH of the second acidic environment is less than or equal to 6. In some embodiments, the pH of the second acidic environment is 4 to 6, particularly 4.5 to 5.5, particularly 4.8 to 5.2, particularly 4.9 to 5.1, and more particularly about 5. In general, for the first anion that is less acidic or more basic, a second acidic environment with lower pH is selected. In some embodiments, the first anion is a hexafluorophosphate ion, and the pH of the second acidic environment is 4 to 6, particularly 4.5 to 5.5, particularly 4.8 to 5.2, particularly 4.9 to 5.1, and more particularly about 5. In some embodiments, the pH of the first acidic environment is the same as that of the second acidic environment.

In some embodiments, the second acidic environment is obtained by the first salt in the second solvent. In some embodiments, the second acidic environment is obtained by the first salt and an acid consisting of the first anion and a hydrogen ion in the second solvent. In some embodiments, further adding a reductant to the first solution, and in particular, adding a reductant to the first solution before, after or at the same time as the second reactant is added to the first solution. That is, in some embodiments, the second acidic environment is reductive, and in particular strongly reductive. Due to the strong reducing capacity of the non-covalent dimer cation of the present application, addition of the reductant is conductive to reduction a proportion of the non-covalent dimer cation of the present application that is oxidized during reaction, and is conductive to increase of the yield of the salt of the present application. In some embodiments, the reductant comprises at least one or more selected from a group consisting of vitamin C, vitamin E, and a derivative of vitamin C or vitamin E.

### Formation of the Salt of the Present Application

A first base is added to the second solution to remove the first acid (H a A in formula (B1)) in the second solution. Then, the salt of the present application is obtained.

In some embodiments, the salt of the present application comprises the non-covalent dimer cation of the present application and the first anion. For example, the salt of the present application may be shown as formula (B).

In some embodiments, the first base comprises or consists of, but is not limited to, at least one selected from a group consisting of ammonia water, sodium hydroxide, potassium hydroxide, and triethanolamine. Those skilled in the art should understand that any base that can adjust the pH and make a solution alkaline without causing a further redox reaction can be taken as the first base. In some embodiments, the first base is ammonia water. In some embodiments, the first base is sodium hydroxide. In some embodiments, the first base is potassium hydroxide. In some embodiments, the first base is triethanolamine.

In some embodiments, the adding of the first base to the second solution adjusts the pH of the second solution to be alkaline. In some embodiments, alkalinity refers to that the pH is 8 or more, particularly 8 to 10, particularly 8.5 to 9.5, particularly 8.8 to 9.2, and more particularly about 9.

In some embodiments, after the first base is added to the second solution, further performing purification to obtain the salt of the present application. In some embodiments, the purification comprises dialysis.

In some embodiments, the dialysis is performed by using a membrane with molecular weight cut off of 50 Da to 500 Da, and in particular about 100 Da.

### Use of the Non-Covalent Dimer Cation of the Present Application or the Salt of the Present Application

In some embodiments, use of the cation or salt of the present application in antioxidation is provided. In some embodiments, use of the cation or salt of the present application in preparation of an antioxidant is provided. In some embodiments, a method of antioxidation, comprising administering the cation or salt of the present application, is provided. In some embodiments, the cation or salt of the present application for use in antioxidation is provided. In some embodiments, the antioxidant is a drug for inhibiting, reducing or reversing oxidative stress in human or animal cells.

In some embodiments, use of the cation or salt of the present application in preparation of a drug for inhibiting, reducing or reversing oxidative stress in human or animal cells is provided. In some embodiments, use of the cation or salt of the present application in preparation of a drug for inhibiting, reducing, and reversing oxidative stress in human or animal cells is provided. In some embodiments, a method for inhibiting, reducing or reversing oxidative stress in human or animal cells, comprising administering the cation or salt of the present application, and in particular, to human or animal cells, is provided. In some embodiments, the cation or salt of the present application for use in inhibition, reductions or reversion of oxidative stress in human or animal cells is provided.

In some embodiments, use of the cation or salt of the present application in treatment of an oxidative stress-associated disease or symptom is provided. In some embodiments, use of the cation or salt of the present application in preparation of a drug for treating an oxidative stress-associated disease or symptom is provided. In some embodiments, a method for treating an oxidative stress-associated disease or symptom, comprising administering the cation or salt of the present application, and in particular, to a patient with an oxidative stress-associated disease or symptom, is provided. In some embodiments, the cation or salt of the present application for use in treatment of an oxidative stress-associated disease or symptom is provided.

In some embodiments, the oxidative stress-associated disease or symptom comprises, but not limited to, aging, inflammation, overweight, obesity, cancer, tumor, hepatopathy, Alzheimer's disease, arterial hypertension, atherosclerosis, cardiovascular disease, diabetes, hypercholesterolemia, Parkinsonian syndrome, chronic fatigue syndrome, ischemia reperfusion injury, neurodegenerative disease, or ultraviolet-induced damage. In some embodiments, hepatopathy comprises, but is not limited to, fatty liver, and in particular, non-alcoholic fatty liver disease or alcoholic fatty liver disease. In some embodiments, the oxidative stress-associated disease or symptom is non-alcoholic fatty liver disease.

In some embodiments, the oxidative stress-associated disease comprises diseases associated with increase of reactive oxygen species, increase of malondialdehyde, reduction of glutathione, reduction of glutathione peroxidase, reduction of superoxide dismutase, and/or reduction of adenosine triphosphate. In some embodiments, the oxidative stress-associated disease comprises a disease associated with increase of reactive oxygen species. In some embodiments, the oxidative stress-associated disease comprises a disease associated with increase of malondialdehyde. In some embodiments, the oxidative stress-associated disease comprises a disease associated with reduction of glutathione. In some embodiments, the oxidative stress-associated disease comprises a disease associated with reduction of glutathione peroxidase. In some embodiments, the oxidative stress-associated disease comprises a disease associated with reduction of superoxide dismutase. In some embodiments, the oxidative stress-associated disease comprises a disease associated with reduction of catalase. In some embodiments, the oxidative stress-associated disease comprises a disease associated with reduction of adenosine triphosphate.

### EXAMPLES

In the following examples, if the compound therein, including the first reactant therein, the second reactant therein, the cation therein, or the salt therein, has chirality that is not explicitly specified, the compound may thus be a chiral mixture such as a racemic compound.

### Example 1

A salt of Example 1 is specifically shown as formula (1) or formula (1′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, CQ⁶ is hydroxyl, n=1, m=1, k=1, and X is hexafluorophosphate ion.

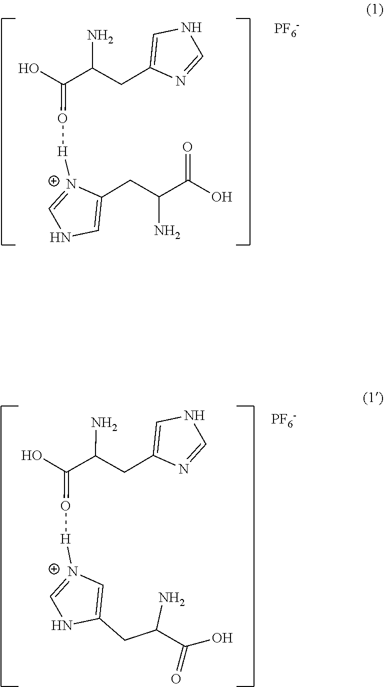

### Method of Preparation

The preparation method of the salt of this example is as follows. Wherein:
the first reactant is 2-amino-3-(1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product or the Intermediate

The hydrochloride of the first reactant of this example is white crystal, with a yield of about 0.8 g and the yield of about 80%.

The yield of the salt of this example is about 1.2 g, and the yield is about 83%.

Conducting a high-resolution mass spectrometry to the salt of this example, and obtains that the cation of this example, (C₁₂H₁₉N₆O₄)⁺, has m/z=311.1.

### Example 2

A salt of Example 2 is specifically shown as formula (2) or formula (2′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=2, and X is hexafluorosilicate ion.

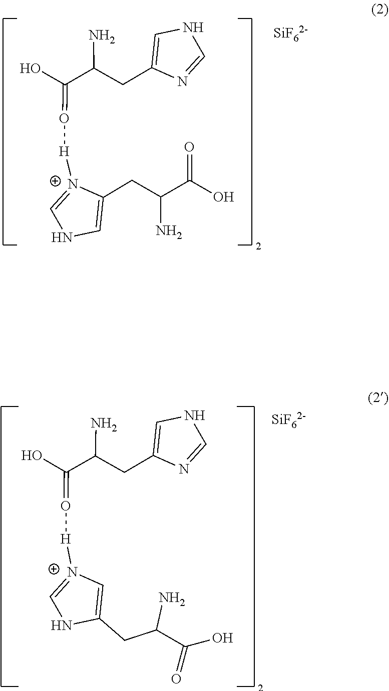

### Method of Preparation

The preparation method of the salt of this example is as follows. Wherein:
the first reactant is 2-amino-3-(1H-imidazol-4-yl)propionic acid;
the second reactant is 2-amino-3-(1H-imidazol-4-yl)propionic acid;
the first salt is ammonium hexafluorosilicate, i.e. the first anion is hexafluorosilicate ion.

At 37° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 37° C. for 12 h, to obtain a first aqueous solution, including a protonated hydrochloride cation of the first reactant, and the first anion.

At 37° C., putting 0.65 g of second reactant into the first aqueous solution and stirring gently for 36 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product or the Intermediate

The hydrochloride of the first reactant of this example is white crystal, with a yield of about 0.8 g and the yield of about 80%.

The yield of the salt of this example is about 0.9 g, and the yield is about 62%.

Conducting a high-resolution mass spectrometry to the salt of this example, and obtains that the cation of this example, (C₁₂H₁₉N₆O₄)+, has m/z=311.1.

### Example 3

A salt of Example 3 is specifically shown as formula (3) or formula (3′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is chloride ion.

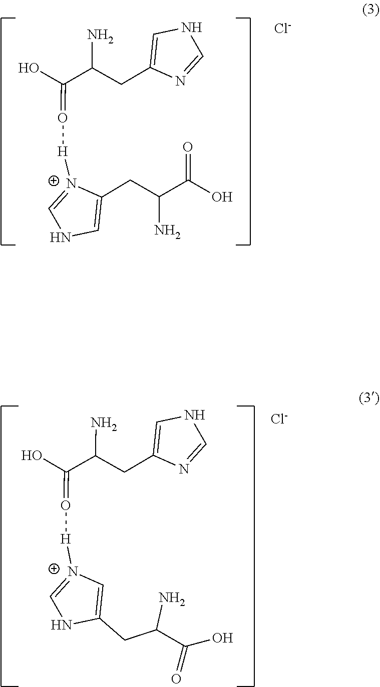

### Method of Preparation

The preparation method of the salt of this example is as follows. Wherein:
the first reactant is 2-amino-3-(1H-imidazol-4-yl)propionic acid;
the second reactant is 2-amino-3-(1H-imidazol-4-yl)propionic acid;
the first salt is ammonium chloride, i.e. the first anion is chloride ion.

At 60° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 2 g of first salt of this example, and stirring at 60° C. for 6 h, to obtain a first aqueous solution, including a protonated hydrochloride cation of the first reactant, and the first anion.

At 60° C., putting 0.65 g of second reactant into the first aqueous solution and stirring gently for 48 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product or the Intermediate

The hydrochloride of the first reactant of this example is white crystal, with a yield of about 0.8 g and the yield of about 80%.

The yield of the salt of this example is about 0.6 g, and the yield is about 41%.

Conducting a high-resolution mass spectrometry to the salt of this example, and obtains that the cation of this example, (C₁₂H₉N₆O₄)⁺, has m/z=311.1.

### Example 4

A salt of Example 4 is specifically shown as formula (4) or formula (4′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is hydrogen, Q⁶ is hydroxyl, n=1, m=1, k=1, and X is chloride ion.

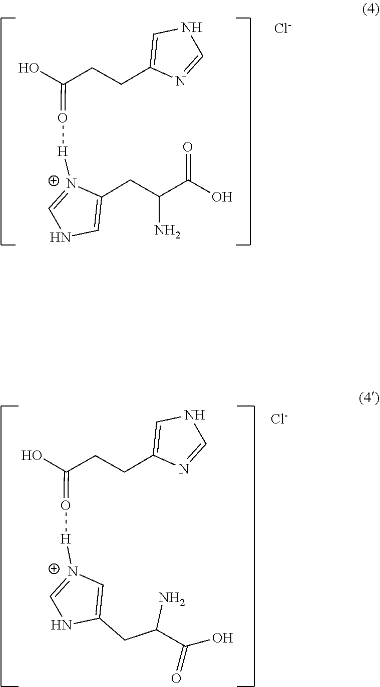

### Method of Preparation

The preparation method of the salt of this example is as follows. Wherein:
the first reactant is 2-amino-3-(1H-imidazol-4-yl)propionic acid;
the second reactant is 3-(1H-imidazol-4-yl)propionic acid;
the first salt is ammonium chloride, i.e. the first anion is chloride ion.

At 4° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 2 g of first salt of this example, and stirring at 4° C. for 6 h, to obtain a first aqueous solution, including a protonated hydrochloride cation of the first reactant, and the first anion.

At 4° C., putting 0.46 g of second reactant into the first aqueous solution and stirring gently for 48 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product or the Intermediate

The hydrochloride of the first reactant of this example is white crystal, with a yield of about 0.8 g and the yield of about 80%.

The yield of the salt of this example is about 0.6 g, and the yield is about 41%.

Conducting a high-resolution mass spectrometry to the salt of this example, and obtains that the cation of this example, (C₁₂H₁₈N₅O₄)⁺, has m/z=296.1.

### Example 5

A salt of Example 5 is specifically shown as formula (5) or formula (5′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 3-aminopropionamido, Q⁶ is hydroxyl, n=1, m=1, k=2, and X is sulfate ion.

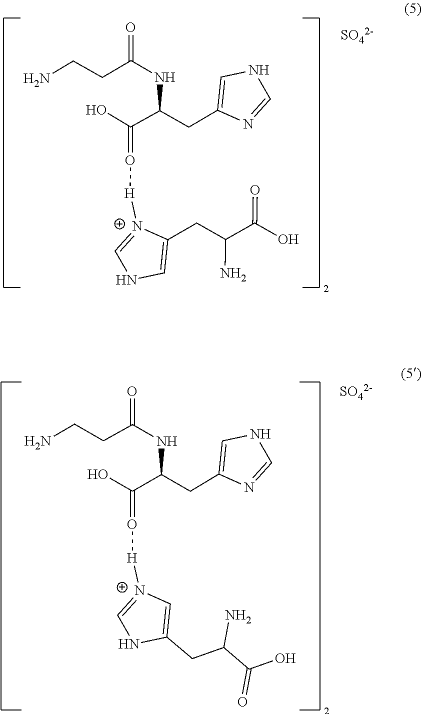

### Method of Preparation

The preparation method of the salt of this example is as follows. Wherein:
the first reactant is 2-amino-3-(1H-imidazol-4-yl)propionic acid;
the second reactant is β-alanyl-L-histidine (i.e. carnosine);
the first salt is ammonium sulfate, that is, a first anion is a sulfate ion.

At 4° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 3 g of first salt of this example, and stirring at 4° C. for 6 h, to obtain a first aqueous solution, including a protonated hydrochloride cation of the first reactant, and the first anion.

At 4° C., putting 0.94 g of second reactant into the first aqueous solution and stirring gently for 48 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product or the Intermediate

The hydrochloride of the first reactant of this example is white crystal, with a yield of about 0.8 g and the yield of about 80%.

The yield of the salt of this example is about 0.6 g, and the yield is about 41%.

Conducting a high-resolution mass spectrometry to the salt of this example, and obtains that the cation of this example, (₁₅H₂₄N₇O₅)⁺, has m/z=382.4.

### Example 6

A salt of Example 6 is specifically shown as formula (6) or formula (6′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydroxyl, Q² is hydrogen, Q³ is hydrogen, Q⁴ is hydrogen, Q⁵ is amino, Q⁶ is hydroxyl, n=1, m=1, k=1, and X is hexafluorophosphate ion.

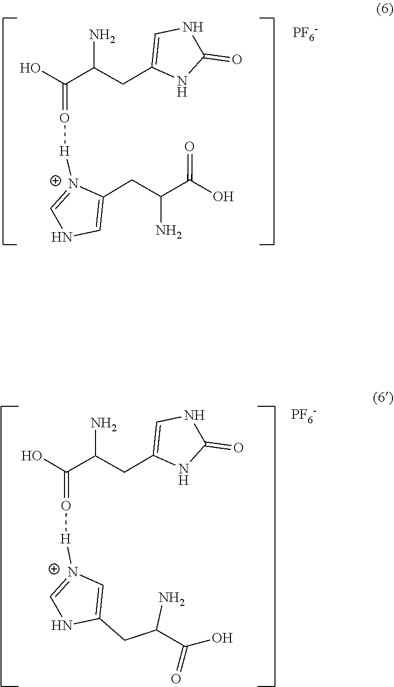

### Method of Preparation

The preparation method of the salt of this example is as follows. Wherein:
the first reactant is 2-amino-3-(1H-imidazol-4-yl)propionic acid;
the second reactant is 2-amino-3-(2-oxo-2,3-dihydro-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the product

Conducting a high-resolution mass spectrometry to the salt of this example, and obtains that the cation of this example, (C₁₂H₁₉N₆O₅)⁺, has m/z=327.2.

### Example 7

A salt of Example 7 is specifically shown as formula (7) or formula (7′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is amino, Q⁶ is hydroxyl, n=1, m=1, k=1, and X is hexafluorophosphate ion.

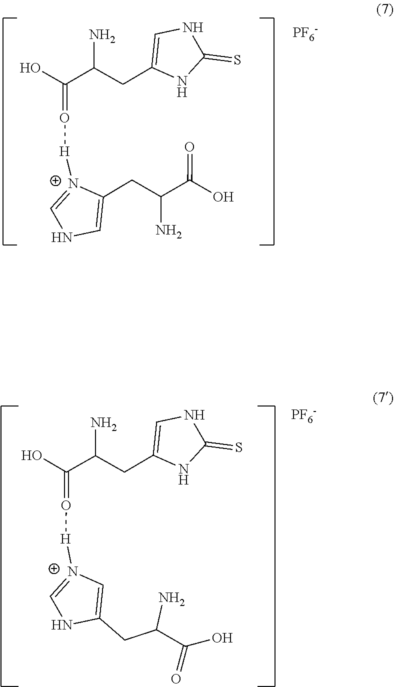

### Method of Preparation

The preparation method of the salt of this example is as follows. Wherein:
the first reactant is 2-amino-3-(1H-imidazol-4-yl)propionic acid;
the second reactant is 2-amino-3-(2-thio-2,3-dihydro-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the product

Conducting a high-resolution mass spectrometry to the salt of this example, and obtains that the cation of this example, (C₁₂H₁₉N₆O₄S)⁺, has m/z=343.4.

### Example 8

A salt of Example 8 is specifically shown as formula (8) or formula (8′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is hydrogen, Q³ is hydrogen, Q⁴ is hydrogen, Q⁵ is trimethylammonium, Q⁶ is oxido, n=1, m=1, k=1, and X is hexafluorophosphate ion.

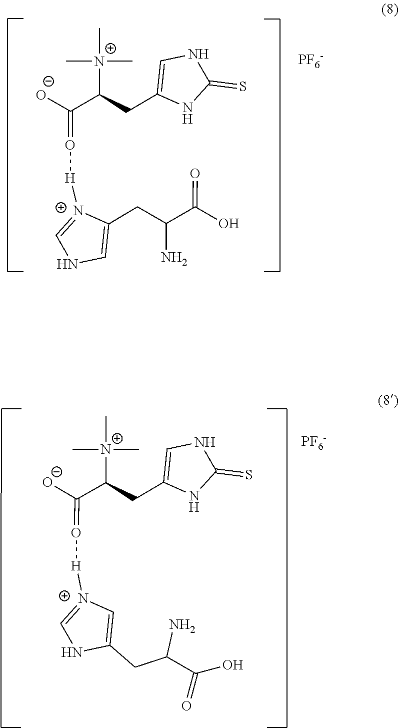

### Method of Preparation

The preparation method of the salt of this example is as follows. Wherein:
the first reactant is 2-amino-3-(1H-imidazol-4-yl)propionic acid;
the second reactant is (S)-3-(2-thio-2,3-dihydro-1H-imidazol-4-yl)-2-(trimethylammonium)propionate;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₅N₆O₄S)⁺, has m/z=385.5.

### Example 9

A salt of Example 9 is specifically shown as formula (9) or formula (9′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is sulfhydryl, Q² is triphenylmethyl, Q³ is hydrogen, Q⁴ is hydrogen, Q⁵ is amino, Q⁶ is methoxy, n=1, m=1, k=1, and X is hexafluorophosphate ion.

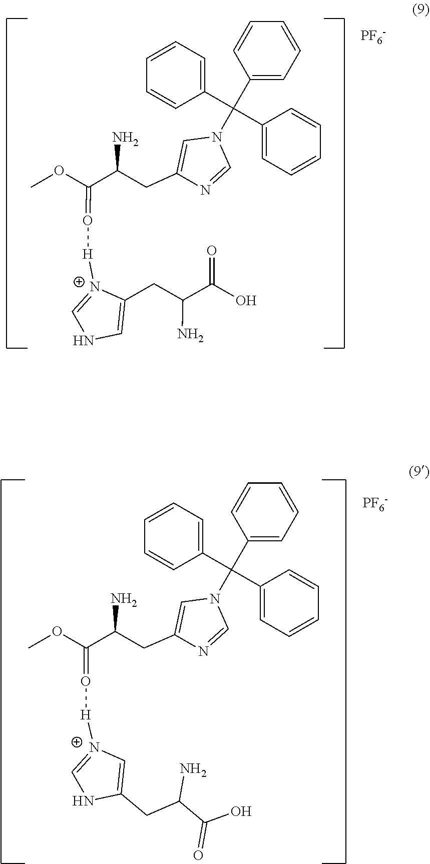

### Method of Preparation

The preparation method of the salt of this example is as follows. Wherein:
the first reactant is 2-amino-3-(1H-imidazol-4-yl)propionic acid;
the second reactant is methyl (S)-2-amino-3-(1-trityl-1H-imidazol-4-yl)propionate (i.e. methyl N^{τ}-trityl-L-histidine);
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₃₂H₃₅N₆O₄)⁺, has m/z=567.7.

### Example 10

A salt of Example 10 is specifically shown as formula (10) or formula (10′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is methyl, Q⁴ is hydrogen, Q⁵ is hydrogen, Q⁶ is hydroxyl, n=1, m=0, k=1, and X is hexafluorophosphate ion.

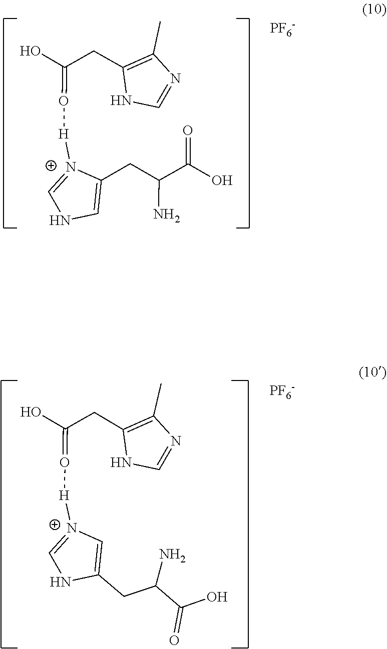

### Method of Preparation

The preparation method of the salt of this example is as follows. Wherein:
the first reactant is 2-amino-3-(1H-imidazol-4-yl)propionic acid;
the second reactant is 2-(5-methyl-1H-imidazol-4-yl)acetic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₈N₅O₄)⁺, has m/z=296.4.

### Example 11

A salt of Example 11 is specifically shown as formula (11) or formula (11′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 2-aminoacetamido, Q⁶ is N² -lysino, n=1, m=1, k=1, and X is hexafluorophosphate ion.

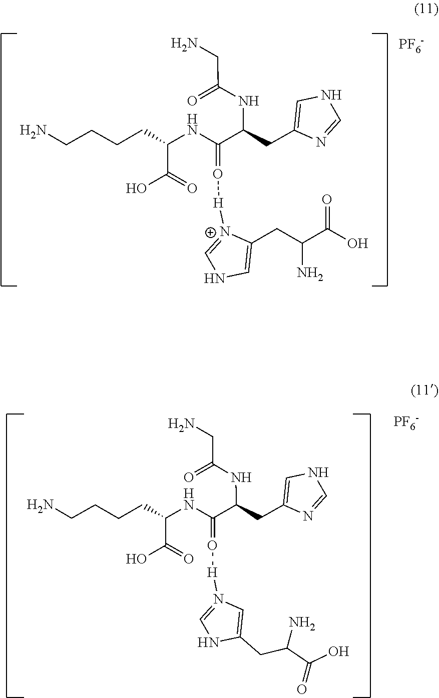

### Method of Preparation

The preparation method of the salt of this example is as follows. Wherein:
the first reactant is 2-amino-3-(1H-imidazol-4-yl)propionic acid;
the second reactant is N² -(N-glycyl-L-histidyl)-L-lysine;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₂₀H₃₄N₉O₆)⁺, has m/z=496.6.

### Example 12

A salt of Example 12 is specifically shown as formula (12) or formula (12′). That is, R¹ is hydrogen, R² is hydrogen, R³ is hydrogen, R⁴ is hydrogen, R⁵ is amino, R⁶ is hydroxyl, R⁷ is —C(═O)—R⁶, Q²¹is hydrogen, Q² is hydrogen, Q³ is hydrogen, Q⁴ is hydrogen, Q⁵ is pyroglutamylamino, Q⁶ is glycinoprolino, n=1, m=1, k=1, and X is hexafluorophosphate ion.

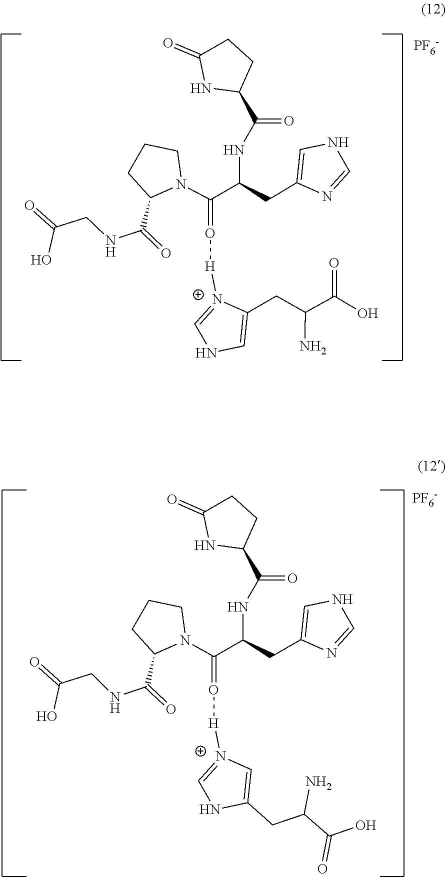

### Method of Preparation

The preparation method of the salt of this example is as follows. Wherein:
the first reactant is 2-amino-3-(1H-imidazol-4-yl)propionic acid;
the second reactant is L-pyroglutamyl-L-histidyl-L-prolylglycine;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

Detection of the product

Conducting a high-resolution mass spectrometry to the salt of this example, and obtains that the cation of this example, (C₂₄H₃₄N₉O₈)⁺, has m/z=576.6.

### Example 13

A salt of Example 13 is specifically shown as formula (13) or formula (13′). That is, R¹ is hydrogen, R² is hydrogen, R³ is hydrogen, R⁴ is hydrogen, R⁵ is amino, R⁷ is hydrogen, CV is hydrogen, Q² is hydrogen, Q³ is hydrogen, Q⁴ is hydrogen, Q⁵ l is amino, Q⁶ is hydroxyl, n=1, m=1, k=1, and X is hexafluorophosphate ion.

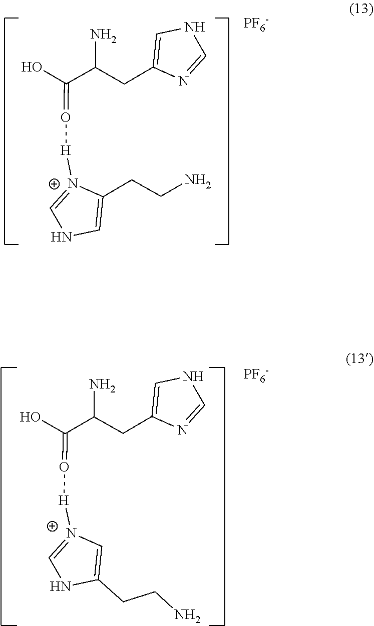

### Method of Preparation

The preparation method of the salt of this example is as follows. Wherein:
the first reactant is 2-(1H-imidazol-4-yl)ethyl-1-amine (i.e. histamine);
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₈N₅O₄)⁺, has m/z=296.1.

### Example 14

A salt of Example 14 is specifically shown as formula (14) or formula (14′). That is, R¹ is hydroxyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

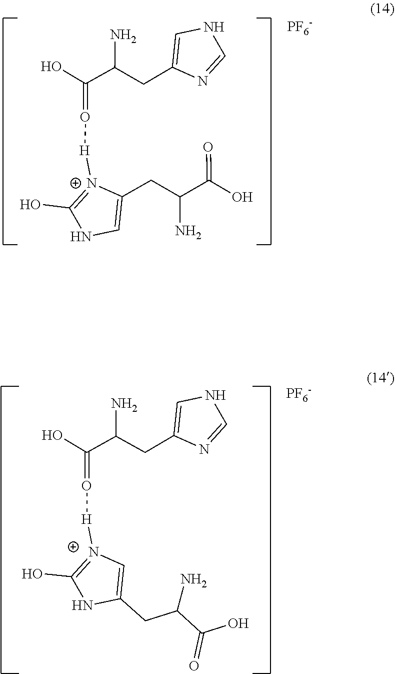

### Method of Preparation

The preparation method of the salt of this example is as follows. Wherein:
the first reactant is 2-amino-3-(2-oxo-2,3-dihydro-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₉N₆O₅)⁺, has m/z=327.2.

### Example 15

A salt of Example 15 is specifically shown as formula (15) or formula (15′). That is, R¹ is sulfhydryl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen Q⁵ is amino, Q⁶ is hydroxyl, n=1, m=1, k=1, and X is hexafluorophosphate ion.

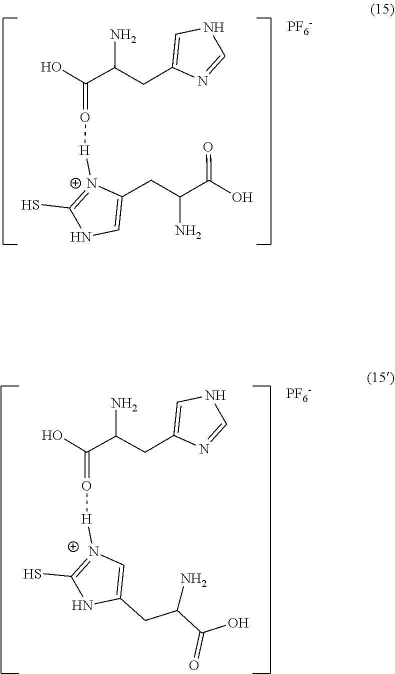

### Method of Preparation

The preparation method of the salt of this example is as follows. Wherein:
the first reactant is 2-amino-3-(2-thio-2,3-dihydro-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₉N₆O₄S)⁺, has m/z=343.4.

### Example 16

A salt of Example 16 is specifically shown as formula (16) or formula (16′). That is, R¹ is hydrogen, R² is hydrogen, R³ is hydrogen, R⁴ is hydrogen, R⁵ is 3-aminopropionamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

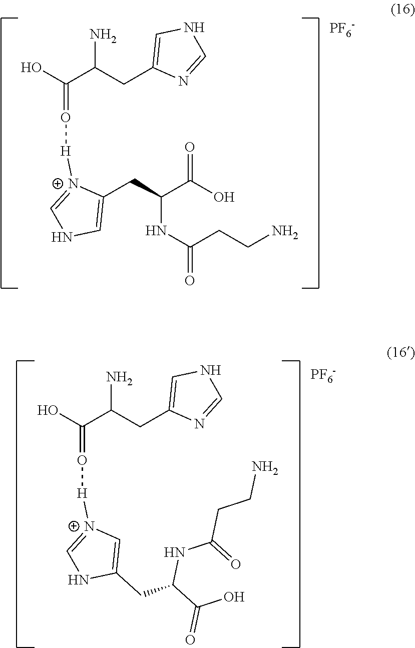

### Method of Preparation

The preparation method of the salt of this example is as follows. Wherein:
the first reactant is β-alanyl-L-histidine (carnosine);
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₄N₇O₅)⁺, has m/z=382.4.

### Example 17

A salt of Example 17 is specifically shown as formula (17). That is, R¹ is hydrogen, R² is triphenylmethyl, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

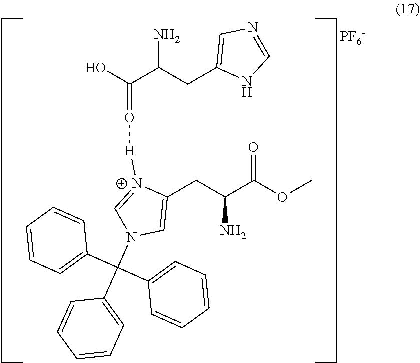

### Method of Preparation

The preparation method of the salt of this example is as follows. Wherein:
the first reactant is methyl (S)-2-amino-3-(1-trityl-1H-imidazol-4-yl)propionate (i.e. methyl N_{τ} trityl-L-histidine);
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₃₂H₃₅N₆O₄)⁺, has m/z=567.7.

### Example 18

A salt of Example 18 is specifically shown as formula (18) or formula (18′). That is, R¹ is sulfhydryl, R² is hydrogen, R³ is hydrogen, R⁴ is hydrogen, R⁵ is trimethylammonium, R⁶ is oxido, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

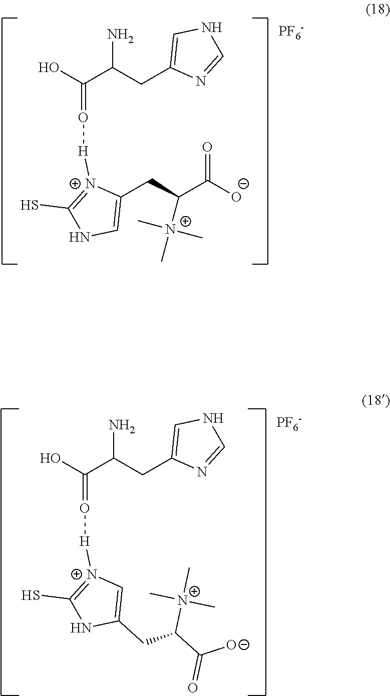

### Method of Preparation

The preparation method of the salt of this example is as follows. Wherein:
the first reactant is (S)-3-(2-thio-2,3-dihydro-1H-imidazol-4-yl) -2-(trimethylammonium)propionate;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₅N₆O₄S)⁺, has m/z=385.5.

### Example 19

A salt of Example 19 is specifically shown as formula (19) or formula (19′). That is, R¹ is hydrogen, R² is hydrogen, R³ is methyl, R⁴ is hydrogen, R⁵ is hydrogen, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=0, m=1, k=1, and X is hexafluorophosphate ion.

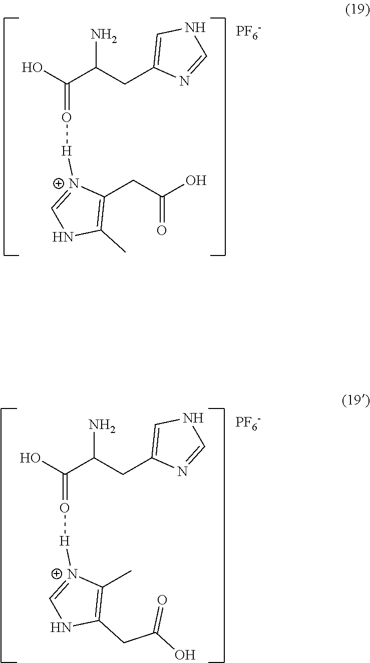

### Method of Preparation

The preparation method of the salt of this example is as follows. Wherein:
the first reactant is 2-(5-methyl-1H-imidazol-4-yl)acetic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₈N₅O₄)⁺, has m/z=296.4.

### Example 20

A salt of Example 20 is specifically shown as formula (20) or formula (20′). That is, R¹ is hydrogen, R² is hydrogen, R³ is hydrogen, R⁴ is hydrogen, R⁵ is 2-aminoacetamido, R⁶ is N² -lysino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

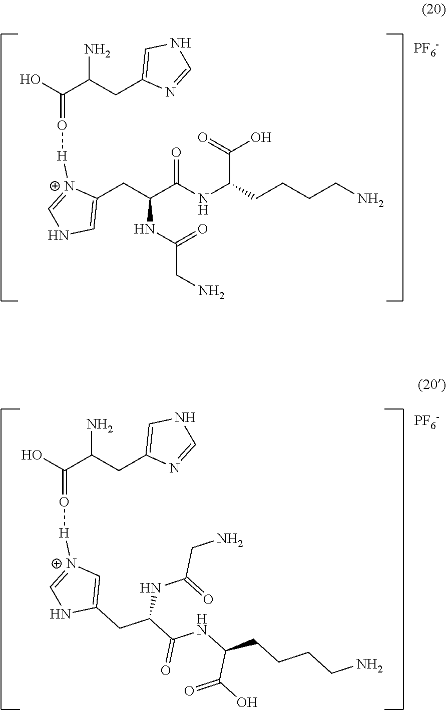

### Method of Preparation

The preparation method of the salt of this example is as follows. Wherein:
the first reactant is N² -(N--glycyl-L-histidyl)-L-lysine;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C201-134N906)+, has m/z=496.6.

### Example 21

A salt of Example 21 is specifically shown as formula (21) or formula (21′). That is, R¹ is hydrogen, R² is hydrogen, R³ is hydrogen, R⁴ is hydrogen, R⁵ is pyroglutamylamino, R⁶ is glycinoprolino, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

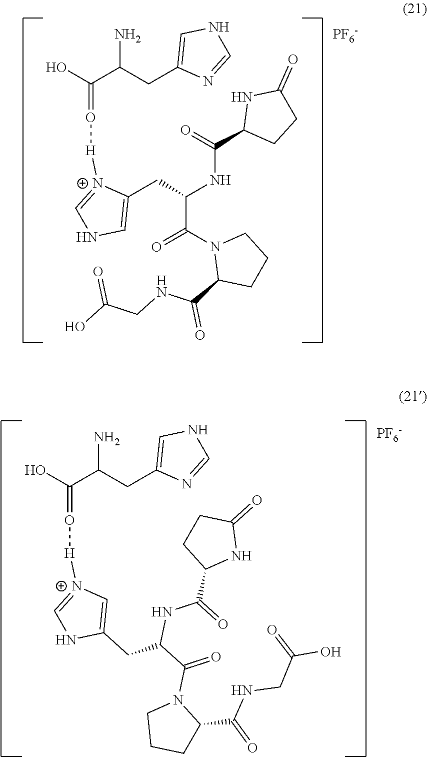

### Method of Preparation

The preparation method of the salt of this example is as follows. Wherein:
the first reactant is L-pyroglutamyl-L-histidyl-L-prolylglycine;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₂₄H₃₄N₉O₈)⁺, has m/z=576.6.

### Example 22

A salt of Example 22 is specifically shown as formula (22) or formula (22′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is amino, Q⁵ is methyl, Q⁶ is hydroxyl, n=1, m=1, k=1, and X is hexafluorophosphate ion.

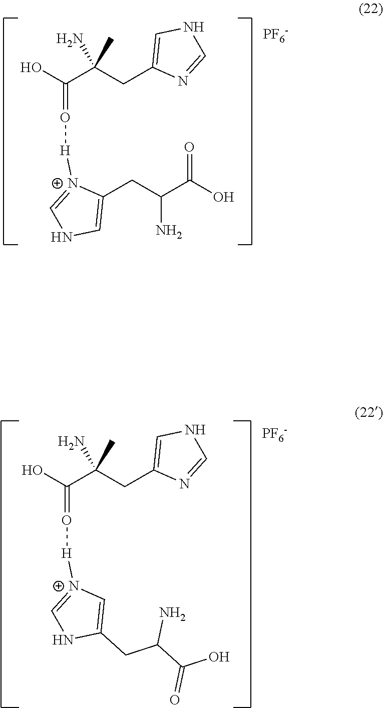

### Method of Preparation

The preparation method of the salt of this example is as follows. Wherein:
the first reactant is 2-amino-3-(1H-imidazol-4-yl)propionic acid;
the second reactant is (R)-2-amino-3-(1H-imidazol-4-yl)-2-methylpropionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₃H₂₁N₆O₄)⁺, has m/z=325.4.

### Example 23

A salt of Example 23 is specifically shown as formula (23) or formula (23′). That is, R¹ is hydrogen, R² is hydrogen, R³ is hydrogen, R⁴ is amino, R⁵ is methyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

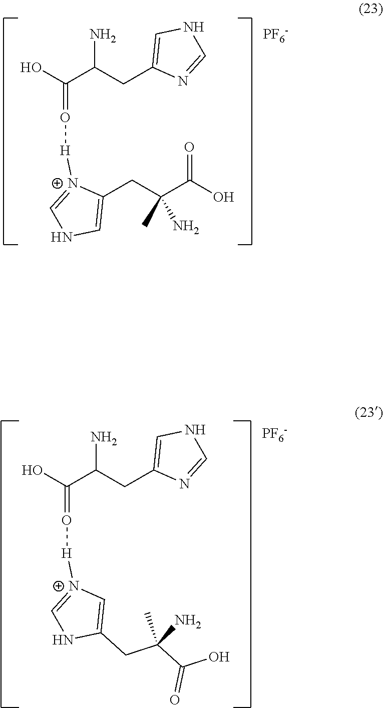

### Method of Preparation

The preparation method of the salt of this example is as follows. Wherein:
the first reactant is (R)-2-amino-3-(1H-imidazol-4-yl)-2-methylpropionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₃H₂₁N₆O₄)⁺, has m/z=325.4.

### Example 24

A salt of Example 24 is specifically shown as formula (24) or formula (24′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is amino, Q² is hydrogen, Q³ is hydrogen, Q⁴ is hydrogen, Q⁵ is amino, Q⁶ is hydroxyl, n=1, m=1, k=1, and X is hexafluorophosphate ion.

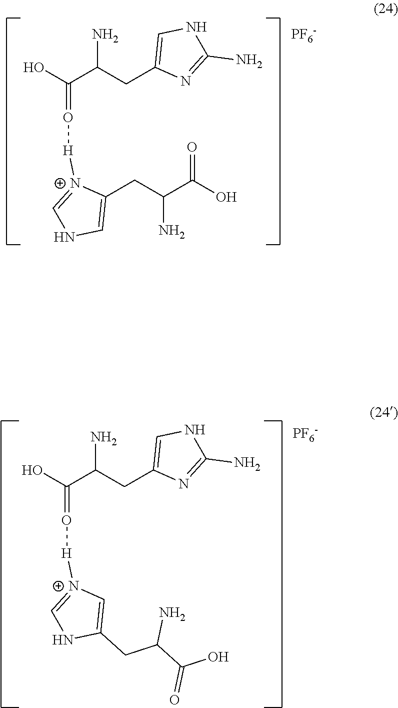

### Method of Preparation

The preparation method of the salt of this example is as follows. Wherein:
the first reactant is 2-amino-3-(1H-imidazol-4-yl)propionic acid;
the second reactant is 2-amino-3-(2-amino-1H-imidazole-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₂₀N₇O₄)⁺, has m/z=326.3.

### Example 25

A salt of Example 25 is specifically shown as formula (25) or formula (25′). That is, R¹ is amino, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

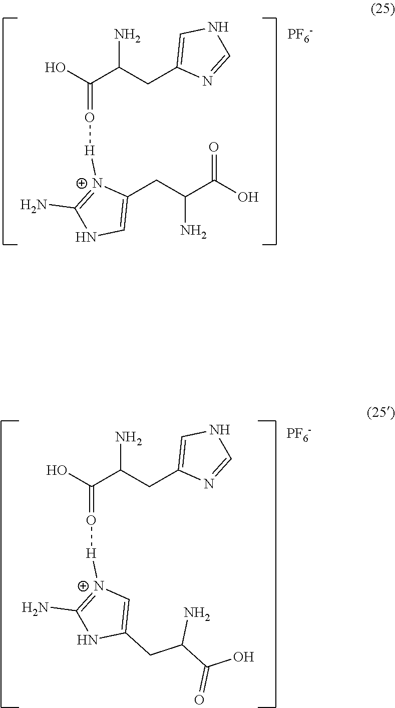

### Method of Preparation

The preparation method of the salt of this example is as follows. Wherein:
the first reactant is 2-amino-3-(2-amino-1H-imidazole-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₂₀N₇O₄)⁺, has m/z=326.3.

### Example 26

A salt of Example 26 is specifically shown as formula (26) or formula (26′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is methoxy, n=1, m=1, k=1, and X is hexafluorophosphate ion.

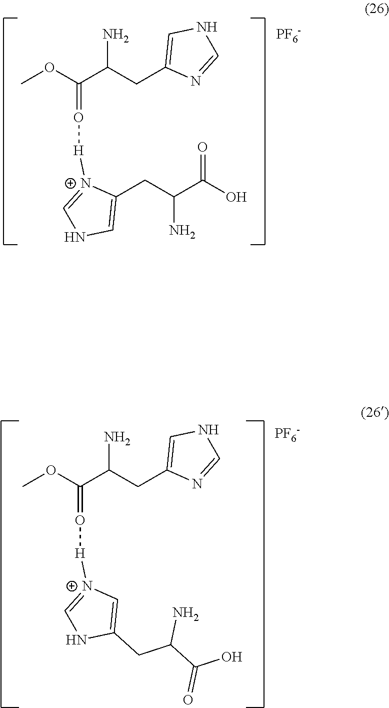

### Method of Preparation

The preparation method of the salt of this example is as follows. Wherein:
the first reactant is 2-amino-3-(1H-imidazol-4-yl)propionic acid;
the second reactant is methyl 2-amino-3-(1H-imidazol-4-yl)propionate;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₃H₂₁N₆O₄)⁺, has m/z=325.2.

### Example 27

A salt of Example 27 is specifically shown as formula (27) or formula (27′). That is, R¹ is hydrogen, R² is hydrogen, R³ is hydrogen, R⁴ is hydrogen, R⁵ is amino, R⁶ is methoxy, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

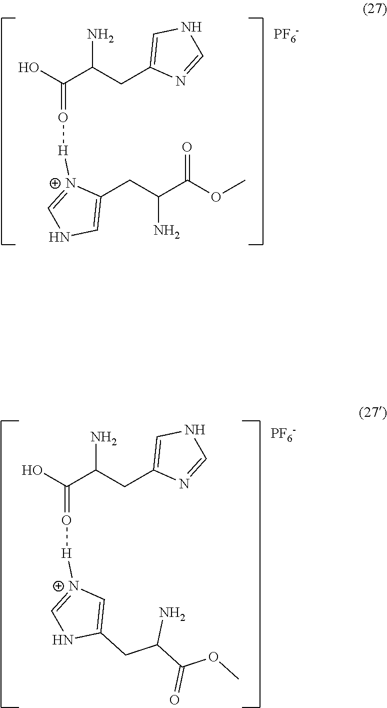

### Method of Preparation

The preparation method of the salt of this example is as follows. Wherein:
the first reactant is methyl 2-amino-3-(1H-imidazol-4-yl)propionate;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₃H₂₁N₆O₄)⁺, has m/z=325.2.

### Example 28

A salt of Example 28 is specifically shown as formula (28) or formula (28′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is hydrazino, Q⁶ is hydroxyl, n=1, m=1, k=1, and X is hexafluorophosphate ion.

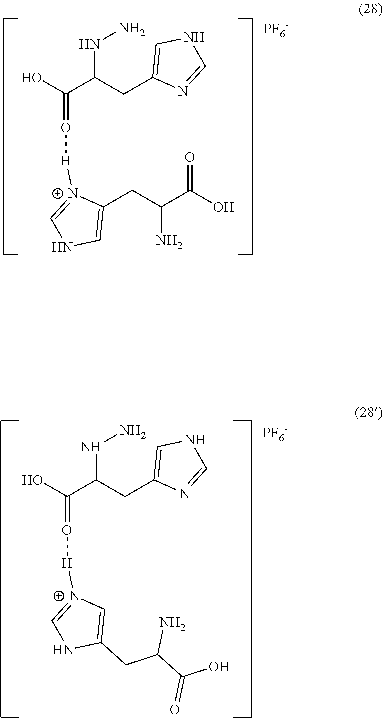

### Method of Preparation

The preparation method of the salt of this example is as follows. Wherein:
the first reactant is 2-amino-3-(1H-imidazol-4-yl)propionic acid;
the second reactant is aminohistidine;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₀N₇O₄)⁺, has m/z=326.4.

### Example 29

A salt of Example 29 is specifically shown as formula (29) or formula (29′). That is, R¹ is hydrogen, R² is hydrogen, R³ is hydrogen, R⁴ is hydrogen, R⁵ is hydrazino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

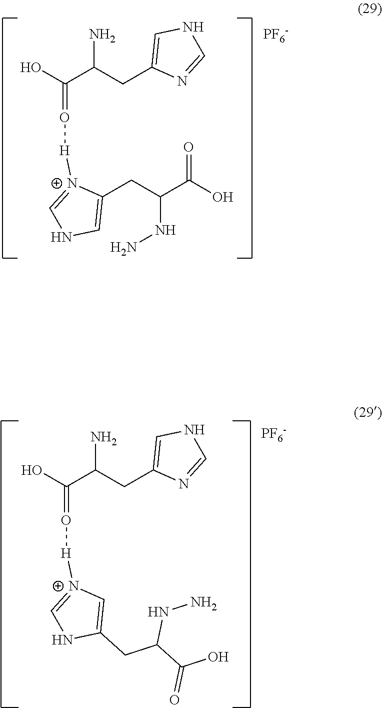

### Method of Preparation

The preparation method of the salt of this example is as follows. Wherein:
the first reactant is aminohistidine;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₀N₇O₄)⁺, has m/z=326.4.

### Example 30

A salt of Example 30 is specifically shown as formula (30) or formula (30′). That is, R¹ is hydrogen, R² is hydrogen, R³ is hydrogen, R⁴ is hydrogen, R⁵ is amino, R⁶ is hydroxyl, R⁷ is —C(═O)—R⁶, CV is hydrogen, Q² is methyl, Q³ is hydrogen, Q⁴ is hydrogen, Q⁵ is amino, Q⁶ is hydroxyl, n=1, m=1, k=1, and X is hexafluorophosphate ion.

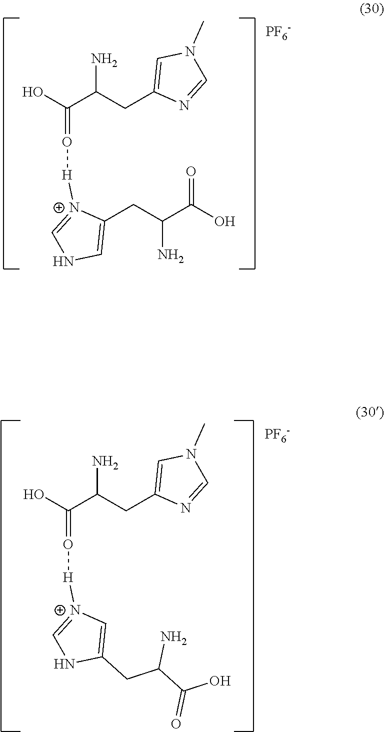

### Method of Preparation

The preparation method of the salt of this example is as follows. Wherein:
the first reactant is 2-amino-3-(1H-imidazol-4-yl)propionic acid;
the second reactant is 2-amino-3-(1-methyl-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₃H₂₁N₆O₄)⁺, has m/z=325.2.

### Example 31

A salt of Example 31 is specifically shown as formula (31). That is, R¹ is hydrogen, R² is methyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶² is hydroxyl, n=1, m=1, k=1, and X is hexafluorophosphate ion.

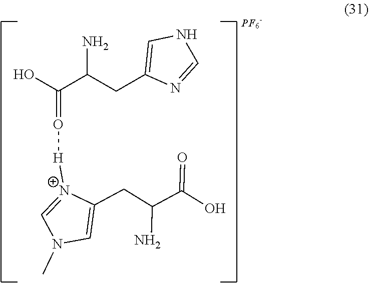

### Method of Preparation

The preparation method of the salt of this example is as follows. Wherein:
the first reactant is 2-amino-3-(1-methyl-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₃H₂₁N₆O₄)⁺, has m/z=325.2.

### Example 32

A salt of Example 32 is specifically shown as formula (32) or formula (32′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is acetamido, Q⁶ is hydroxyl, n=1, m=1, k=1, and X is hexafluorophosphate ion.

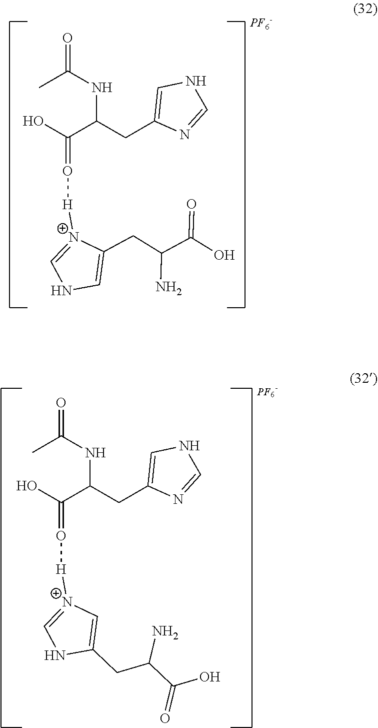

### Method of Preparation

The preparation method of the salt of this example is as follows. Wherein:
the first reactant is 2-amino-3-(1H-imidazol-4-yl)propionic acid;
the second reactant is 2-acetamido-3-(1H-imidazole-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₄H₂₁N₆O₅)⁺, has m/z=353.3.

Example 33

A salt of Example 33 is specifically shown as formula (33) or formula (33′). That is, R¹ is hydrogen, R² is hydrogen, R³ is hydrogen, R⁴ is hydrogen, R⁵ is acetamido, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

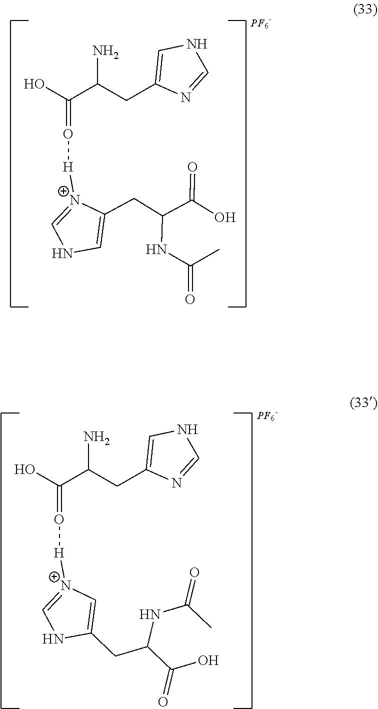

### Method of Preparation

The preparation method of the salt of this example is as follows. Wherein:
the first reactant is 2-acetamido-3-(1H-imidazole-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₄H₂₁N₆O₅)⁺, has m/z=353.3.

### Example 34

A salt of Example 34 is specifically shown as formula (34) or formula (34′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is sulfhydryl, Q⁴ is hydrogen, Q⁵ is amino, Q⁶ is hydroxyl, n=1, m=1, k=1, and X is hexafluorophosphate ion.

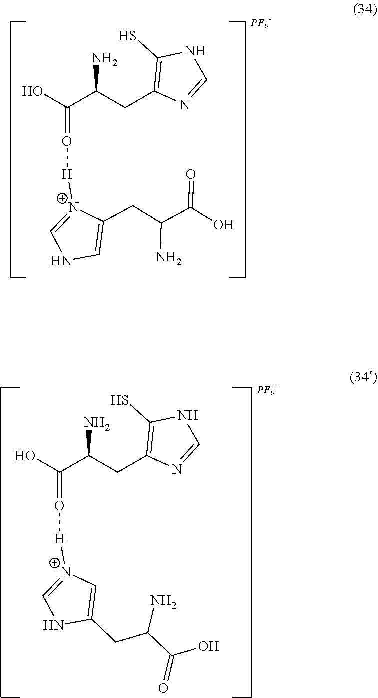

### Method of Preparation

The preparation method of the salt of this example is as follows. Wherein:
the first reactant is 2-amino-3-(1H-imidazol-4-yl)propionic acid;
the second reactant is (S)-2-amino-3-(5-mercapto-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₉N₆O₄S)⁺, has m/z=343.4.

### Example 35

A salt of Example 35 is specifically shown as formula (35) or formula (35′). That is, R¹ is hydrogen, R² is hydrogen, R³ is sulfhydryl, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

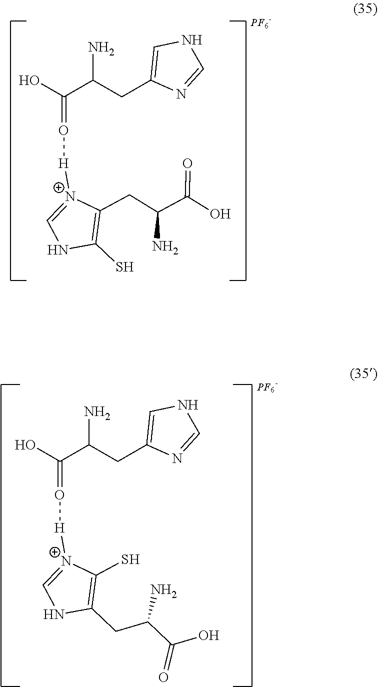

### Method of Preparation

The preparation method of the salt of this example is as follows. Wherein:
the first reactant is (S)-2-amino-3-(5-mercapto-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₁₉N₆O₄S)⁺, has m/z=343.4.

### Example 36

A salt of Example 36 is specifically shown as formula (36) or formula (36′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is phenyl, Q² is hydrogen, Q³ is hydrogen, Q⁴ is hydrogen, Q⁵ is amino, Q⁶ is hydroxyl, n=1, m=1, k=1, and X is hexafluorophosphate ion.

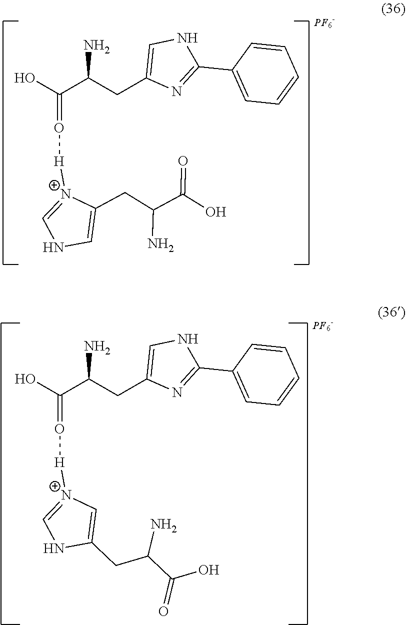

### Method of Preparation

The preparation method of the salt of this example is as follows. Wherein:
the first reactant is 2-amino-3-(1H-imidazol-4-yl)propionic acid;
the second reactant is (S)-2-amino-3-(2-phenyl-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₈H₂₄N₆O₄)⁺, has m/z=388.2.

### Example 37

A salt of Example 37 is specifically shown as formula (37) or formula (37′). That is, R¹ is phenyl, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

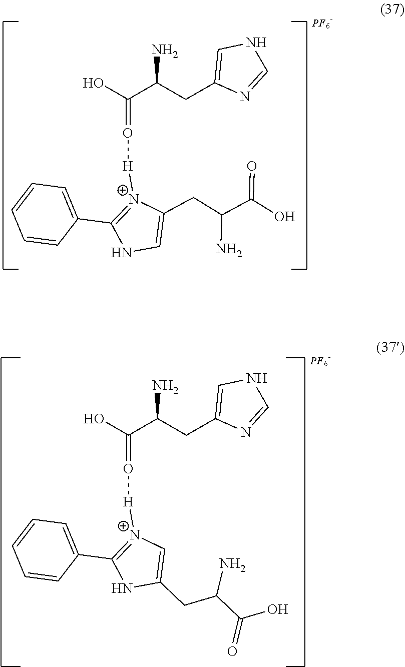

### Method of Preparation

The preparation method of the salt of this example is as follows. Wherein:
the first reactant is (S)-2-amino-3-(2-phenyl-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₈H₂₄N₆O₄)⁺, has m/z=388.2.

### Example 38

A salt of Example 38 is specifically shown as formula (38) or formula (38′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is 3-aminopropionamido, Q⁶ is hydroxyl, n=1, m=1, k=1, and X is hexafluorophosphate ion.

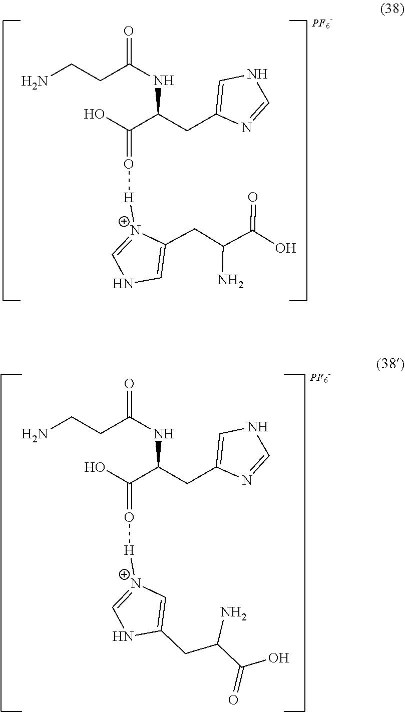

### Method of Preparation

The preparation method of the salt of this example is as follows. Wherein:
the first reactant is 2-amino-3-(1H-imidazol-4-yl)propionic acid;
the second reactant is β-alanyl-L-histidine (i.e. carnosine);
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₄N₇O₅)⁺, has m/z=382.4.

### Example 39

A salt of Example 39 is specifically shown as formula (39) or formula (39′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is propyl, Q³ is hydrogen, Q⁴ is hydrogen, Q⁵ is amino, Q⁶ is hydroxyl, n=1, m=1, k=1, and X is hexafluorophosphate ion.

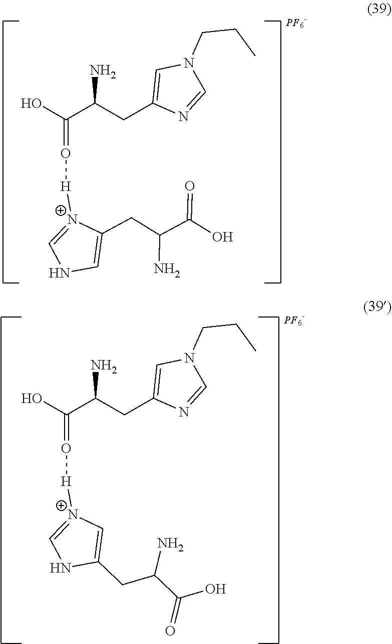

### Method of Preparation

The preparation method of the salt of this example is as follows. Wherein:
the first reactant is 2-amino-3-(1H-imidazol-4-yl)propionic acid;
the second reactant is (S)-2-amino-3-(1-propyl-1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₆N6O₄)⁺, has m/z=353.5.

### Example 40

A salt of Example 40 is specifically shown as formula (40). That is, R¹ is hydrogen, R² is propyl, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

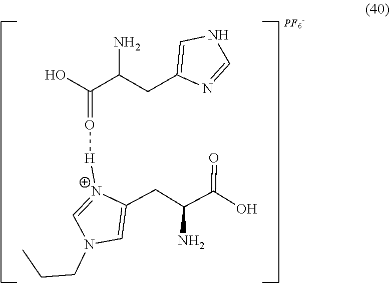

### Method of Preparation

The preparation method of the salt of this example is as follows. Wherein:
the first reactant is (S)-2-amino-3-(1-propyl-1H-imidazol-4-yl)propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₅H₂₆N6O₄)⁺, has m/z=353.5.

### Example 41

A salt of Example 41 is specifically shown as formula (41) or formula (41′). That is, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is pyrrolyl, Q⁶ is hydroxyl, n=1, m=1, k=1, and X is hexafluorophosphate ion.

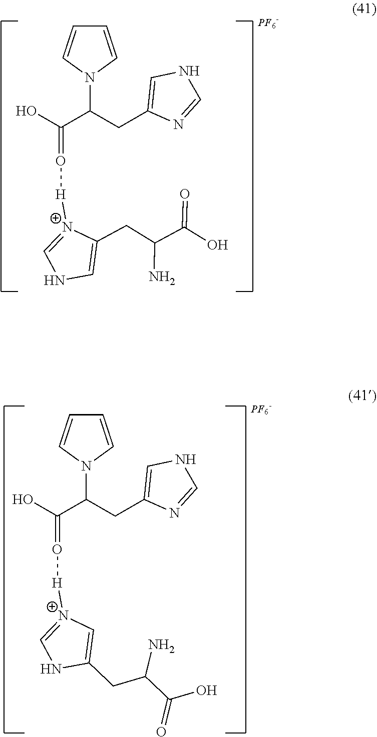

### Method of Preparation

The preparation method of the salt of this example is as follows. Wherein:
the first reactant is 2-amino-3-(1H-imidazol-4-yl)propionic acid;
the second reactant is 2-amino-3-(1H-imidazol-4-yl)-2-(pyrrol-1-yl)-propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₆H₂₁N₆O₄)⁺, has m/z=361.4.

### Example 42

A salt of Example 42 is specifically shown as formula (42) or formula (42′). That is, R¹ is hydrogen, R² is hydrogen, R³ is hydrogen, R⁴ is hydrogen, R⁵ is pyrrolyl, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

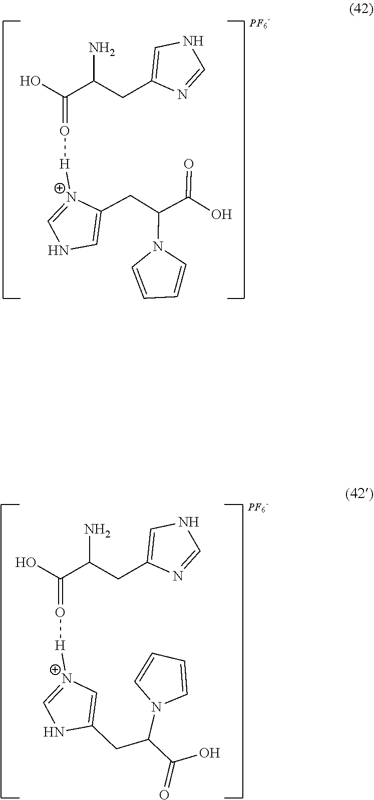

### Method of Preparation

The preparation method of the salt of this example is as follows. Wherein:
the first reactant is 2-amino-3-(1H-imidazol-4-yl)-2-(pyrrol-1-yl)-propionic acid;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₆H₂₁N₆O₄)⁺, has m/z=361.4.

### Example 43

A salt of Example 43 is specifically shown as formula (43) or formula (43′). That is, R¹ is hydrogen, R² is hydrogen, R³ is hydrogen, R⁴ is hydrogen, R⁵ is amino, R⁷ is hydroxyl, Q¹ is hydrogen, Q² is hydrogen, Q³ is hydrogen, Q⁴ is hydrogen, Q⁵ is amino, Q⁶ is hydroxyl, n=1, m=1, k=1, and X is hexafluorophosphate ion.

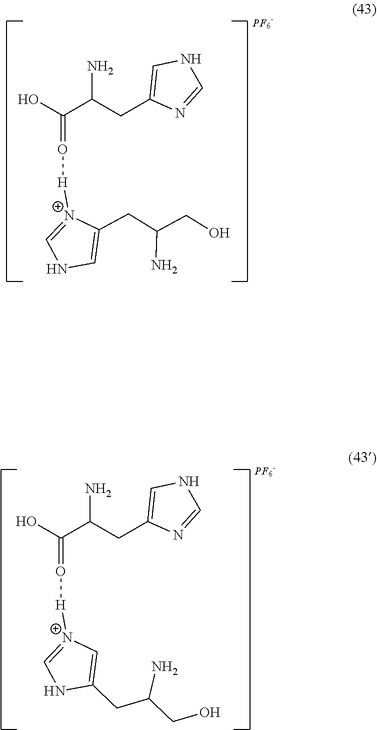

### Method of Preparation

The preparation method of the salt of this example is as follows. Wherein:
the first reactant is 2-amino-3-(1H-imidazol-4-yl)propanol;
the second reactant is 2-amino-3-(1H-imidazol-4-yl)propionic acid;
the first salt is ammonium hexafluorophosphate, i.e. the first anion is hexafluorophosphate ion.

At 25° C., placing 1 g of first reactant into 10 mL of 2 M hydrochloric acid and stirring intensely until the first reactant was completely dissolved. Heating to 85° C. and keeping at this temperature for 30 min, and concentrating under reduced pressure to 2 mL. Cooling the solution, and crystals were precipitated, the crystals were washed 3 times with an ethanol solution and dried, to obtain a hydrochloride of the first reactant.

Placing 0.8 g of hydrochloride of the first reactant into 10 mL of deionized water, stirring intensely until the hydrochloride of the first reactant was completely dissolved, adding 4 g of first salt of this example, and stirring at 25° C. for 6 h, to obtain a first aqueous solution, including a protonated hydrochloride cation of the first reactant, and the first anion.

At 25° C., putting 0.65 g of second reactant into the first aqueous solution and stirring gently for 24 h, to obtain a second aqueous solution comprising the cation of this example. Adjusting the pH of the second aqueous solution to 9. Completely dialyzing with a dialysis bag with molecular weight cut-off of 100 Da. Drying to obtain a product, i.e. the salt of this example.

### Detection of the Product

Conducting a high-resolution mass spectrometry to the salt of this example, and obtains that the cation of this example, (C₁₂H₂₁N₆O₃)⁺, has m/z=297.4.

### Oxygen Species Scavenging Experiments in Hydrogen Peroxide Model

The salts of Example 1, Example 16, and Example 13 were taken as the compound of experimental groups, and histidine was taken as the compound of control group. For each compound, different final concentrations were set, which were 0.025 g/mL, 0.05 g/mL, 0.1 g/mL, 0.2 g/mL, 0.4 g/mL, and 0.8 g/mL, respectively.

20 μL of each experimental or control compound was added to 80 pi of 0.5 M hydrogen peroxide aqueous solution to obtain an aqueous solution in which the final concentration of hydrogen peroxide was 0.4 M, and the final concentration of experimental or control compound was as above. After the aqueous solution was shaken at 37° C. for 1 h, the content of remaining hydrogen peroxide was measured by potassium permanganate titration.

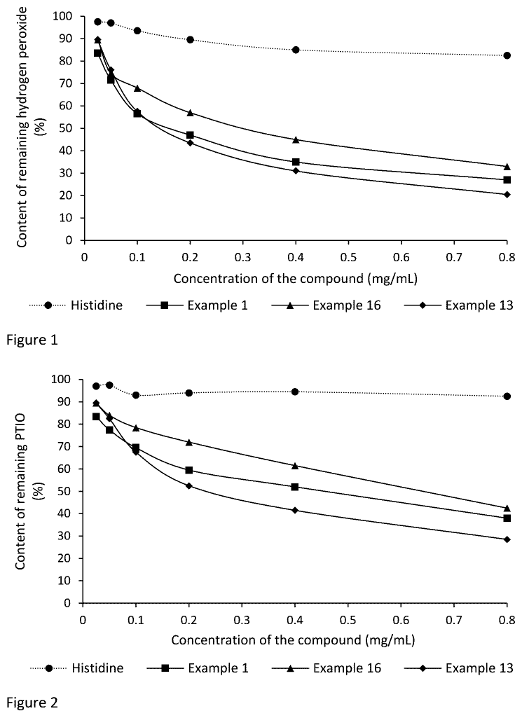

Results are shown in FIG. 1. The x-axis represents the concentration of the experimental or control compound, and the y-axis represents the normalized content of remaining hydrogen peroxide, wherein the initial content of hydrogen peroxide is defined as 100%.

As shown in FIG. 1, when the concentration is at least 0.025 m/mL, the non-covalent dimer cation of the present application can scavenge oxygen species represented by hydrogen peroxide more efficiently compared to histidine, and the scavenging efficiency is at least 3 to 5 times that of histidine; furthermore, with the increase of the concentration, the oxygen species scavenging effect is improved.

Oxygen Species Scavenging Experiments in PTIO Model

The salts of Example 1, Example 16, and Example 13 were taken as the compound of experimental groups, and histidine was taken as the compound of control group. For each compound, different final concentrations were set, which were 0.025 g/mL, 0.05 g/mL, 0.1 g/mL, 0.2 g/mL, 0.4 g/mL, and 0.8 g/mL, respectively.

20 μL of each experimental or control compound was added to 80 μL of 0.1 mg/mL PTIO aqueous solution to obtain an aqueous solution in which the final concentration of PTIO was 0.08 mg/mL, and the final concentration of each experimental or control compound was as above. After the aqueous solution was shaken at 37° C. for 1 h, the content of remaining PTIO were measured by measuring the absorbance at the wavelength of 560 nm.

Results are shown in FIG. 2. The x-axis represents the concentration of the experimental or control compound, and the y-axis represents the normalized content of remaining PTIO, wherein the initial content of PTIO is defined as 100%.

As shown in FIG. 2, when the concentration is at least 0.025 g/mL, the non-covalent dimer cation of the present application can scavenge oxygen species represented by PTIO more efficiently compared to histidine, and the scavenging efficiency is at least 7 to 10 times that of histidine; furthermore, with the increase of the concentration, the oxygen species scavenging effect is improved.

Cell Models of Palmitate-Induced Non-Alcoholic Fatty Liver Disease

Palmitate is a kind of saturated fatty acid, and is often used to mimic the pathogenesis of non-alcoholic fatty liver disease in vitro. In this experiment, a model of non-alcoholic fatty liver disease was built by using palmitate at the cellular level, and the salt of the present application or another control compound was added, to obtain technical effects of the non-covalent dimer cation of the present application on the model of non-alcoholic fatty liver disease.

The salts of Example 1, Example 16, and Example 13 were taken as the compound of experimental groups, vitamin E was taken as the compound of control group. A blank group (without palmitate) and a model group (with palmitate but with neither the salt of the present application nor vitamin E) were also set.

L02 human normal hepatocytes were uniformly inoculated into a 96-well plate, about 5,000 cells per well. The cells were cultured in a complete DMEM for 6 h. After the cells were completely adhered to the wall, all the media were removed, 100 μL of serum-free medium containing 0.05 mg/mL palmitate was added to each well (for the blank group, a serum-free medium without palmitate), and the cells were cultured for 24 h.

Then, all the media were removed, 100 μL of serum-free DMEM containing 0.025 g/mL, 0.05 g/mL, 0.1 g/mL, 0.2 g/mL, 0.4 g/mL or 0.8 g/mL compound of experimental groups or control group was added to each well (for the blank group and the model group, serum-free DMEM without the compound) according to the groups, and the cells were cultured for 24 h.

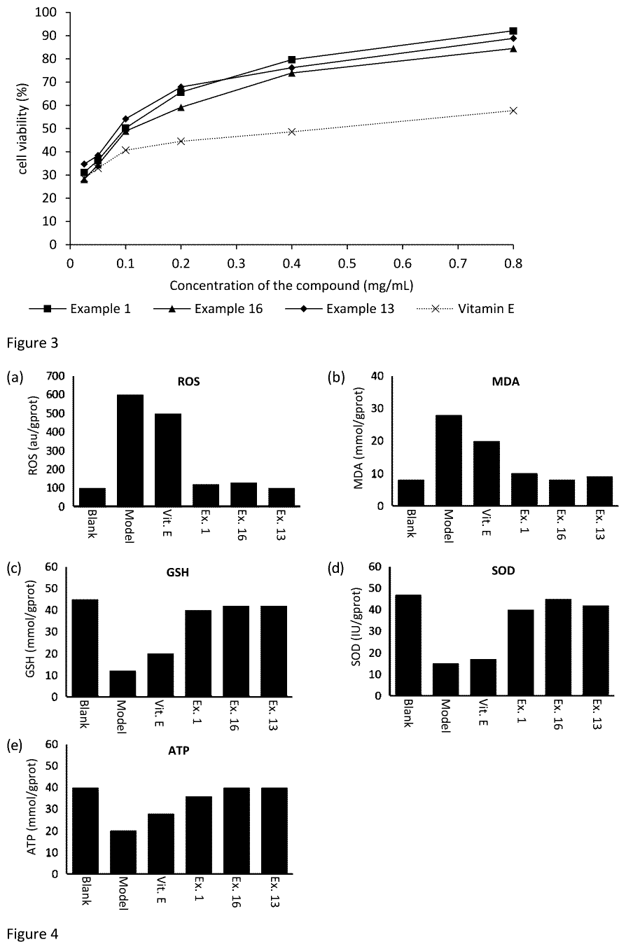

Finally, the cell viability was detected by using CCK-8. Results of the model group show that, after the cells cultured in the medium containing palmitate for 24 h, the cell viability is significantly reduced to only about 30%, and the similar cell viability is maintained during next 24 h of culture. Results of the experimental groups and the control group are shown in FIG. 3. It can be seen that after adding the salt of the present application to the cell culture solution, the higher the concentration thereof, the higher the cell viability, and the cell viability of the group containing the salt of the present application is significantly higher than that of the group containing vitamin E. It indicates that the non-covalent dimer cation of the present application can improve the cell viability by scavenging free radicals and promoting production of energy (ATP). Furthermore, the cation of the present application essentially consists of histidine, analog thereof or derivative thereof, so its biological safety, especially biological safety for human body, is relatively high.

50 μL of RIPA lysis buffer (strong) was added to each well to lyse the cells, and levels of reactive oxygen species, malondialdehyde, glutathione, superoxide dismutase, and adenosine triphosphate in the cells of each group were detected by using corresponding chemical reagents. Results are shown in FIG. 4.

Please refer to part (a) of FIG. 4. Compared with the cells of the blank group, the cells of the model group demonstrate significantly increase in the content of ROS because of the addition of palmitate. The vitamin E treatment can only slightly reduce the level of ROS in the cells. By comparison, after treatment with the non-covalent dimer cation of the present application, the level of ROS in the cells can be significantly reduced to be very close to the level of ROS in the cells of the blank group.

Please refer to part (b) of FIG. 4. Compared with the cells of the blank group, the cells of the model group demonstrate significantly increase in the content of MDA because of the addition of palmitate. The vitamin E treatment can only slightly reduce the level of MDA in the cells. By comparison, after treatment with the non-covalent dimer cation of the present application, the level of MDA in the cells can be significantly reduced to be very close to the level of MDA in the cells of the blank group.

It indicates that the non-covalent dimer cation of the present application has an anti-lipid peroxidation effect.

Please refer to part (c) of FIG. 4. Compared with the cells of the blank group, the cells of the model group demonstrate significantly reduction in the content of GSH because of the addition of palmitate. The vitamin E treatment can only slightly increase the level of GSH in the cells. By comparison, after treatment with the non-covalent dimer cation of the present application, the level of GSH in the cells can be significantly increased to be very close to the level of GSH in the cells of the blank group.

Please refer to part (d) of FIG. 4. Compared with the cells of the blank group, the cells of the model group demonstrate significantly reduction in the content of SOD because of the addition of palmitate. The vitamin E treatment can only slightly increase the level of SOD in the cells. By comparison, after treatment with the non-covalent dimer cation of the present application, the level of SOD in the cells can be significantly increased to be very close to the level of SOD in the cells of the blank group.

Please refer to part (e) of FIG. 4. Compared with the cells in the blank group, the cells of the model group demonstrate significantly reduction in the content of ATP because of the addition of palmitate. The vitamin E treatment can only partially increase the level of ATP in the cells. By comparison, after treatment with the non-covalent dimer cation of the present application, the level of ATP in the cells can be significantly increased to be very close to the level of ATP in the cells of the blank group.

It indicates that the non-covalent dimer cation of the present application can increase the level of endogenous cellular antioxidants (GSH and SOD) in fatty liver damaged cells, increase the content of ATP, and promote cells to restore the viability thereof. The non-covalent dimer cation of the present application not only has antioxidant capacity, but also has play a role in repairing cell functions of liver cells and repairing non-alcoholic fatty liver damage due to the unique spatial structure thereof.

### Cell Models of Carbon tTetrachloride-roduced Hepatotoxicity

Carbon tetrachloride produces significant hepatotoxicity through free radical damage, and is often used to build a model of hepatotoxicity. In this experiment, a model of hepatotoxicity was built by using carbon tetrachloride at the cellular level, and the salt of the present application or another control compound was added, to obtain technical effect of the non-covalent dimer cation of the present application on the model of hepatotoxicity.

The salts of Example 1, Example 16, and Example 13 were taken as the compound of experimental groups, vitamin E was taken as the compound of control group. A blank group (without carbon tetrachloride) and a model group (with carbon tetrachloride but with neither the salt of the present application nor vitamin E) were also set.

L02 human normal hepatocytes were uniformly inoculated into a 96-well plate, about 5,000 cells per well. The cells were cultured in a complete DMEM for 6 h. After the cells were completely adhered to the wall, all the media were removed, 100 μL of serum-free DMEM containing 0.02 mg/mL carbon tetrachloride (for the blank group, a serum-free medium without carbon tetrachloride) was added to each well, and the cells were cultured for 12 h. Then, all the media were removed, 100 μL of serum-free DMEM containing 0.025 g/mL, 0.05 g/mL, 0.1 g/mL, 0.2 g/mL, 0.4 g/mL or 0.8 g/mL compound of experimental groups or control group was added to each well (for the blank group and the model group, serum-free DMEM without the compound) according to the groups, and the cells were cultured for 24 h.

Finally, the cell viability was detected by using CCK-8. Results of the model group show that, after the cells cultured in the medium containing carbon tetrachloride for 12 h, the cell viability is significantly reduced to only about 20%, and the similar cell viability is maintained during next 24 h of culture. Results of the experimental groups and the control group are shown in FIG. 5. It can be seen that after adding the salt of the present application to the cell culture solution, the higher the concentration thereof, the higher the cell viability, and the cell viability of the group containing the salt of the present application is significantly higher than that of the group containing vitamin E. It indicates that the non-covalent dimer cation of the present application can improve the cell viability by scavenging free radicals and promoting production of energy (ATP). Furthermore, the cation of the present application essentially consists of histidine, analog thereof or derivative thereof, so its biological safety, especially biological safety for human body, is relatively high.

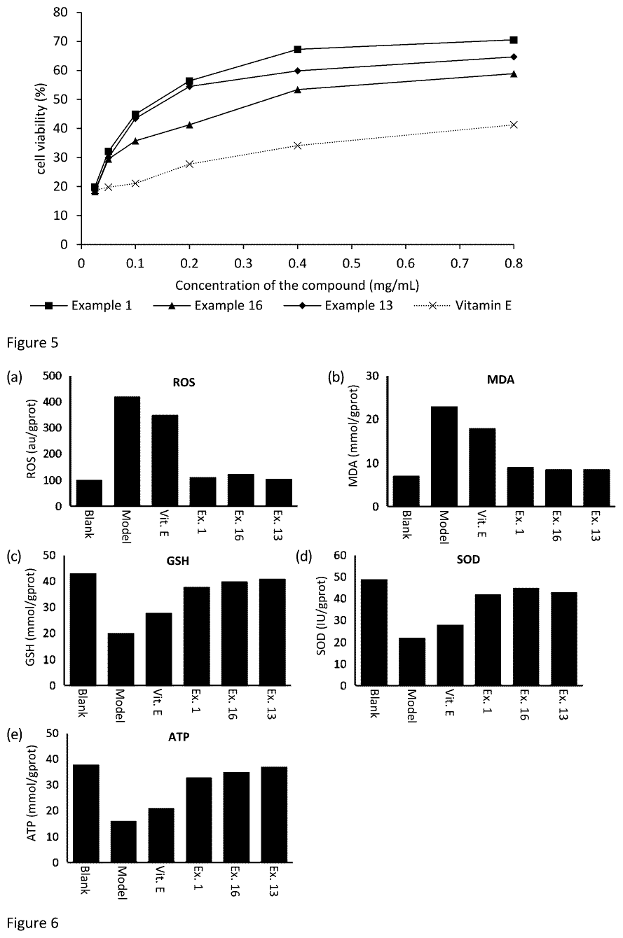

50 μL of RIPA lysis buffer (strong) was added to each well to lyse the cells, and levels of reactive oxygen species, malondialdehyde, glutathione, superoxide dismutase, and adenosine triphosphate in the cells of each group were detected by using corresponding chemical reagents. Results are shown in FIG. 6.

Please refer to part (a) of FIG. 6. Compared with the cells in the blank group, the cells of the model group demonstrate significantly increase in the content of ROS because of the addition of carbon tetrachloride. The vitamin E treatment can only slightly reduce the level of ROS in the cells. By comparison, after treatment with the non-covalent dimer cation of the present application, the level of ROS in the cells can be significantly reduced to be very close to the level of ROS in the cells of the blank group.

Please refer to part (b) of FIG. 6. Compared with the cells in the blank group, the cells of the model group demonstrate significantly increase in the content of MDA because of the addition of carbon tetrachloride. The vitamin E treatment can only slightly reduce the level of MDA in the cells. By comparison, after treatment with the non-covalent dimer cation of the present application, the level of MDA in the cells can be significantly reduced to be very close to the level of MDA in the cells of the blank group.

It indicates that the non-covalent dimer cation of the present application has an anti-lipid peroxidation effect.

Please refer to part (c) of FIG. 6. Compared with the cells in the blank group, the cells of the model group demonstrate significantly reduction in the content of GSH because of the addition of carbon tetrachloride. The vitamin E treatment can only slightly increase the level of GSH in the cells. By comparison, after treatment with the non-covalent dimer cation of the present application, the level of GSH in the cells can be significantly increased to be very close to the level of GSH in the cells of the blank group.

Please refer to part (d) of FIG. 6. Compared with the cells in the blank group, the cells of the model group demonstrate significantly reduction in the content of SOD because of the addition of carbon tetrachloride. The vitamin E treatment can only slightly increase the level of SOD in the cells. By comparison, after treatment with the non-covalent dimer cation of the present application, the level of SOD in the cells can be significantly increased to be very close to the level of SOD in the cells of the blank group.

Please refer to part (e) of FIG. 6. Compared with the cells in the blank group, the cells of the model group demonstrate significantly reduction in the content of ATP because of the addition of carbon tetrachloride. The vitamin E treatment can only partially increase the level of ATP in the cells. By comparison, after treatment with the non-covalent dimer cation of the present application, the level of ATP in the cells can be significantly increased to be very close to the level of ATP in the cells of the blank group.

It indicates that the non-covalent dimer cation of the present application can increase the level of endogenous cellular antioxidants (GSH and SOD) in fatty liver damaged cells, increase the content of ATP, and promote cells to restore the viability thereof. The non-covalent dimer cation of the present application not only has antioxidant capacity, but also has play a role in repairing cell functions of liver cells and repairing non-alcoholic fatty liver damage due to the unique spatial structure thereof.

Based on the foregoing examples and experimental results, it can be seen that:
The cation of the present application exists stably in the environment (for example, exists in the form of the salt of the present application), and also exists stably during mass spectrometry, with a mass spectra peak in corresponding m/z.
The cation of the present application has low effect on cell viability and thus has high biological safety for human body.
The cation of the present application can effectively scavenge oxygen species and has an anti-lipid peroxidation effect, and can increase the level of endogenous cellular antioxidants and plays a cell repairing function.

In some embodiments, the cation or salt of the present application improves the total antioxidant capacity of liver cells or tissues on the one hand, and can repair damaged fatty liver cells or tissues on the other hand, so that the damaged fatty liver cells or tissues approach the morphology and function of normal liver cells, and fatty liver disease is functionally prevented and treated.

The foregoing embodiments are merely preferred embodiments of the present application, and are not intended to limit the scope of protection of the present application. Any non-substantive modifications and substitutions made by t hose skilled in the art on the basis of the present application shall fall within the scope of protection claimed by the present application.

1. A non-covalent dimer cation, wherein the non-covalent dimer cation is a cation represented by formula (A), a tautomer thereof, or a stereoisomer thereof, 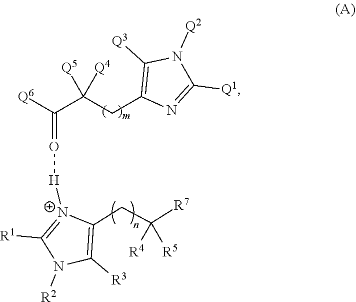
wherein,
the bond “----- ” is a non-covalent bond;
Fe is selected from a group consisting of hydrogen, hydroxymethyl, —C—R⁶, and —C(═O)—R 6 ;
Ft' and CV are each independently selected from a group consisting of hydrogen, hydroxyl, sulfhydryl, amino, methyl, ethyl, and phenyl;
R² and Q² are each independently selected from a group consisting of hydrogen, methyl, ethyl, propyl, phenyl, benzyloxymethyl, and triphenylmethyl;
R³ and Q³ are each independently selected from a group consisting of hydrogen, hydroxyl, sulfhydryl, amino, methyl, ethyl, and phenyl;
R⁴ and Q⁴ are each independently selected from a group consisting of hydrogen, methyl, ethyl, and propyl;
R⁵ and Q⁵ are each independently selected from a group consisting of hydrogen, benzyl, amino, methylamino, hydrazinyl, trimethylammonium, pyrrolyl, and amino further linked with a substituent derived from the C-terminal carbon atom of an amino acid, dipeptide or tripeptide;
R⁶ and Q⁶ are each independently selected from a group consisting of hydroxyl, deprotonated hydroxyl, methoxy, and a substituent derived from the N-terminal nitrogen atom of an amino acid, dipeptide or tripeptide; and
m and n are each independently selected from a group consisting of 0 and 1.

2. The cation according to claim 1, wherein
R⁴ is hydrogen;
R⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino;
R⁶ is selected from a group consisting of hydroxyl, deprotonated hydroxyl, methoxy, N² -lysino, and glycinoprolino; and
n is 1;
and/or
Q⁴ is hydrogen;
Q⁵ is selected from a group consisting of hydrogen, benzyl, amino, methylamino, hydrazinyl, trimethylammonium, pyrrolyl, acetamido, 2-aminoacetamido, 3-aminopropionamido, and pyroglutamylamino;
Q⁶ is selected from a group consisting of hydroxyl, deprotonated hydroxyl, methoxy, N² -lysino, and glycinoprolino; and
m is 1.

3. The cation according to claim 1, wherein,
R⁷ is —C(═O)—R⁶;
n is 1; and
m is 1.

4. The cation according to claim 1, wherein,
R⁴ is hydrogen;
R⁵ is amino or 3-aminopropionamido; and
R⁷ is —C(═O)—R⁶ and R⁶ is hydrogen, or R⁷ is hydrogen;
and/or
Q⁴ is hydrogen;
Q⁵ is amino; and
Q⁶ is hydrogen.

5. The cation according to claim 1, wherein,
R¹ is hydrogen;
R² is hydrogen; and
R³ is hydrogen;
and/or
Q¹ is hydrogen;
Q² is hydrogen; and
Q³ is hydrogen.

6. The cation according to claim 1, wherein,
R¹ is hydrogen;
R² is hydrogen;
R³ is hydrogen;
R⁴ is hydrogen;
R⁵ is amino or 3-aminopropionamido; and
R⁷ is —C(═O)—R⁶ and R⁶ is hydrogen, or R⁷ is hydrogen;
and/or
Q¹ is hydrogen;
Q² is hydrogen;
Q³ is hydrogen;
Q⁴ is hydrogen;
Q⁵ is amino; and
Q⁶ is hydrogen.

7. The cation according to claim 1, wherein, R¹ is hydrogen, R² is hydrogen, R³ is hydrogen, R⁴ is hydrogen, R⁵ is amino, R⁶ is hydroxyl, R⁷ is —C(═O)—R⁶, Q¹ is hydrogen, Q² is hydrogen, Q³ is hydrogen, Q⁴ is hydrogen, Q⁵ is amino, Q⁶ is hydroxyl, n=1, and m =1;
R¹ is hydrogen, R² is hydrogen, R³ is hydrogen, R⁴ is hydrogen, R⁵ is amino, R⁷ is hydrogen, Q¹ is hydrogen, Q² is hydrogen, Q³ is hydrogen, Q⁴ is hydrogen, Q⁵ is amino, Q⁶ is hydroxyl, n=1, and m=1; or
R¹ is hydrogen, R² is hydrogen, R³ is hydrogen, R⁴ is hydrogen, R⁵ is 3-aminopropionamido, R⁶ is hydroxyl, R⁷ is —C(═O)—R⁶, CV is hydrogen, CI' is hydrogen, C1 3 is hydrogen, CI' is hydrogen, C1 5 is amino, Cr is hydroxyl, n=1, and m =1.

8. The cation according to claim 1, wherein,
R⁷ is —C(═O)—R⁶;
R¹ is the same as Q¹;
R² is the same as Q²;
R³ is the same as Q³;
R⁴ is the same as Q⁴;
R⁵ is the same as Q⁵; and
R⁶ is the same as Q⁶.

9. A salt, comprising:
the cation according to claim 1; and
a first anion,
wherein the first anion comprises at least one selected from a group consisting of a fluoride ion, a chloride ion, a bromide ion, an iodide ion, a sulfide ion, a nitrate ion, a sulfate ion, a sulfite ion, a thiosulfate ion, a persulfate ion, a selenate ion, a phosphate ion, a carbonate ion, a hexafluorophosphate ion, a hexafluorosilicate ion, an acetate ion, a sulfonate ion, a benzoate ion, and a polyphosphate ion.

10. The salt according to claim 9, wherein the first anion comprises at least one selected from a group consisting of a hexafluorophosphate ion, a hexafluorosilicate ion, a chloride ion, and a sulfate ion.

11. A preparation method of the cation of claim 1, comprising:
contacting a first reactant with a first acid to obtain a first acid salt of the first reactant;
contacting the first acid salt of the first reactant with a first salt comprising a first anion in a first acidic environment, to obtain a first solution comprising a protonated first acid salt cation of the first reactant and the first anion;
adding a second reactant to the first solution in a second acidic environment, to obtain a second solution; and
adding a first base to the second solution,
wherein, the first reactant comprising a compound represented by formula (C), and the second reactant comprising a compound represented by formula (D),

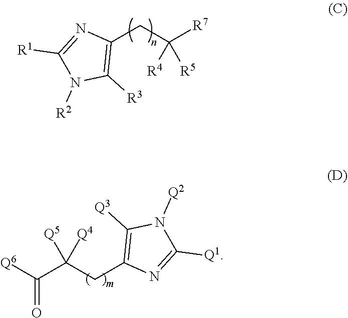

12. The preparation method according to claim 11, wherein
the first acid is selected from a group consisting of hydrochloric acid, hydrobromic acid, and hydroiodic acid; and
the first salt is consisting of a first cation and the first anion, and the first cation is an ammonium ion.

13. The preparation method according to claim 11, wherein
the first reactant is contacted with the first acid at first temperature, and the first temperature is 0° C. to 70° C.;
the first acid salt of the first reactant is contacted with the first salt at second temperature, and the second temperature is 0° C. to 70° C.; and
the second reactant is added to the first solution at third temperature, and the third temperature is 0° C. to 70° C.

14. The preparation method according to claim 11, wherein
a molar ratio of the first reactant to the first acid is 1: 2 to 1: 10;
a molar ratio of the first acid salt of the first reactant to the first salt is 1: 2 to 1: 10; and
a molar ratio of a protonated first reactant cation to the second reactant is 1: 1 to 1: 2.

15. The preparation method according to claim 11, wherein
a pH of the first acidic environment is less than or equal to 6; and
a pH of the second acidic environment is less than or equal to 6.

16. The preparation method according to claim 11, wherein
the first base comprises at least one selected from a group consisting of ammonia water, sodium hydroxide, potassium hydroxide, and triethanolamine; and
the adding the first base to the second solution adjusts the pH of the second solution to be 8 or more.

17. A method of antioxidation comprising administering the cation of claim 1.

18. The method of claim 17, wherein the method comprises inhibiting, reducing or reversing oxidative stress in human or animal cells.

19. The method of claim 17, wherein the method comprises treating an oxidative stress-associated disease or symptom.

20. The method of claim 19, wherein
the disease or symptom is selected from a group consisting of ageing, inflammation, overweight, obesity, cancer, tumor, hepatopathy, Alzheimer's disease, arterial hypertension, atherosclerosis, cardiovascular disease, diabetes, hypercholesterolemia, Parkinsonian syndrome, chronic fatigue syndrome, ischemia reperfusion injury, neurodegenerative disease, and ultraviolet-induced damage; and/or
the disease or symptom is non-alcoholic fatty liver disease.
